(19) United States
(12) Patent Application Publication
(10) Pub. No.: US 2024/0165241 A1
(43) Pub. Date: May 23, 2024

(54) TARGETED DELIVERY OF DRUG MOLECULES WITH DRUG LIGANDS CONJUGATED TO RNA NANOPARTICLE MOTION ELEMENTS

(71) Applicant: Ohio State Innovation Foundation, Columbus, OH (US)

(72) Inventors: Peixuan GUO, Columbus, OH (US); Daniel BINZEL, Columbus, OH (US); Peter BLANCO CARCACHE, Columbus, OH (US); Xin LI, Columbus, OH (US); Congcong XU, Columbus, OH (US)

(21) Appl. No.: 18/170,997

(22) Filed: Feb. 17, 2023

Related U.S. Application Data
(60) Provisional application No. 63/311,717, filed on Feb. 18, 2022.

(51) Int. Cl. A61K 47/54

(52) U.S. Cl. CPC A61K 47/549; A61K 47/545

(57) ABSTRACT

Disclosed herein is a therapeutics structure containing a motion element, an methotrexate (MTX), N—[N—[(S)-1,3-dicarboxypropyl]carbamoyl]-(S)-lysine (DCL), or UAMC-1110 ligand, and a plurality of drugs selected from the group consisting of camptothecin (CPT), paclitaxel (PTX), podophyllotoxin (PTOX), 7-Ethyl-10-hydroxycamptothecin (SN38), BMS1, BMS8, BMS27, BMS242, LY294002, PI3K-IN-20, and methotrexate (MTX).

### CROSS-REFERENCE TO RELATED APPLICATIONS

This application claims benefit of U.S. Provisional Application No. 63/311,717, filed Feb. 18, 2022, which is hereby incorporated herein by reference in its entirety.

### STATEMENT REGARDING FEDERALLY SPONSORED RESEARCH OR DEVELOPMENT

This invention was made with Government Support under Grant Nos. CA207946 and EB019036 awarded by the National Institutes of Health. The Government has certain rights in the invention.

### SEQUENCE LISTING

This application contains a sequence listing filed in ST.26 format entitled “321501-1460 Sequence Listing” created on Jan. 12, 2024, having 45,276 bytes. The content of the sequence listing is incorporated herein in its entirety.

### BACKGROUND

With great promise for therapeutics delivery in a targeted manner, ligand-conjugated nanoparticles have been exploited with controllable physicochemical properties to achieve enhanced tumor targeting (Ramzy, L, et al. Eur J Pharm Sci 2017 104:273-292). For instance, nanoparticulate systems with different size, shape and surface chemistry were reported to have exhibited different in vivo cancer targeting and treatment behavior as a result of the altered biomolecular interactions (Albanese, A, et al. Annu Rev Biomed Eng 2012 14:1-16; Chithrani, B D, et al. Nano Lett 2007 7:1542-1550). To further maximize tumor retention, optimizing binding affinity to target cells remains as a promising regime, as evidenced by the FDA-approved GIVLAARI® which utilized trivalent N-acetylgalactosamine (GaINAc) as targeting ligands for enhanced delivery to hepatocytes (Agarwal, S, et al. Clin Pharmacol Ther. 2020). As the nanoparticles bind to target cells via ligand-receptor recognition, controlling the valency of targeting ligands will potentially govern the cellular uptake efficacy and in vivo biodistribution of the nanoparticles, thus critically impacting the therapeutic outcomes (Weissleder, R, et al. Nat Biotechnol 2005 23:1418-1423).

### SUMMARY

Use of targeted delivery of chemical drug to lower the required dose reduces toxicity and side effects. Achieving specific targeted delivery of therapeutics will increase local concentrations of the therapeutics, thus reducing the required therapeutic dose needed for responses. This targeted delivery of therapeutics will lower the concentrations of formulations delivered to the body, resulting in reduced the toxicities and side-effects.

The use of small chemical drugs as ligand for the delivery of chemical drugs to cancer has not been reported before.

Disclosed herein is the use of self-motion elements to bring and escort the drug into the tumor target. Also disclosed is the construction of therapeutic complexes that include three key components of a motion element, a small chemical drug as ligand, and a payload of chemical drugs for therapy. The motion element enables the complex to find and reach tumor vasculature with high efficiency.

In some embodiments, drugs are directly inculpated into the RNA complex without chemical conjugation. Thus, the RNA complex can be designed to maintain the motion property of the RNA motion element. Production of the therapeutics RNA complex with the chemical drugs becomes part of the RNA complex that has its intrinsic property of unpaired sequence that exercise the motion property. Therefore, the RNA complex carries the chemical drugs while still moving with structural design.

Chemical drug ligand molecules include camptothecin (CPT), paclitaxel (PTX), podophyllotoxin (PTOX), 7-Ethyl-10-hydroxycamptothecin (SN38), BMS1, BMS8, BMS27, BMS242, LY294002, PI3K-IN-20. Chemical ligands include methotrexate (MTX), N—[N—[(S)-1,3-dicarboxypropyl]carbamoyl]-(S)-lysine (DCL), and UAMC-1110.

Therefore, disclosed herein is a therapeutics structure containing a motion element, an MTX ligand and a plurality of drugs selected from the group consisting of CPT, PTX, PTOX, SN38, BMS001, BMS8, BMS27, BMS242, LY294002, PI3K-IN-10, and MTX.

Therefore, disclosed herein is a therapeutics structure containing a motion element, an DCL ligand and a plurality of drugs selected from the group consisting of CPT, PTX, PTOX, SN38, BMS001, BMS8, BMS27, BMS242, LY294002, PI3K-IN-10, and MTX.

Therefore, disclosed herein is a therapeutics structure containing a motion element, an UAMC1110 ligand and a plurality of drugs selected from the group consisting of CPT, PTX, PTOX, SN38, BMS001, BMS8, BMS27, BMS242, LY294002, PI3K-IN-10, and MTX.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1a-1c. Synthesis and characterization of MTX-N₃. FIG. 1a) Scheme showing the modification of MTX with azido group. FIG. 1b) Reverse-phase (RP) HPLC analysis of MTX and purified MTX-N₃. FIG. 1c) Mass spectrometry analysis of MTX and purified MTX-N₃.

FIGS. 2a-2f. Construction and characterization of multiple MTX conjugated RNA nanoparticles. FIG. 2a) Schematic showing the self-assembly of motion element nanoparticles from three RNA oligos. FIG. 2b) Native PAGE analysis of the stepwise assembly of motion element-M-MTX NPs. FIG. 2c) Hydrodynamic size d) Zeta potential e) TGGE characterization of motion element-M-MTX f) Serum stability (n=3, error bars are presented as mean±SD).

FIGS. 3a-3b. Serum protein binding and flow cytometry analysis. FIG. 3a) Serum protein binding profile of motion element-M-MTX nanoparticles (n=3, error bars are presented as mean±SD). FIG. 3b) Flow cytometry analysis of RNA nanoparticles binding to KB cells and HepG2 cells.

FIGS. 4a-4c. In vitro cancer cell internalization and inhibition effect of RNA-MTX nanoparticles. FIG. 4a) Confocal microscopy imaging of cellular uptake of RNA-MTX nanoparticles (blue: nuclei; green: cytoskeleton; red: RNA nanoparticles; scale bar: 50 μm). b) MTT assay showing the in vitro inhibition effect of RNA-MTX complex 72 hrs post-treatment (n=3, error bars are presented as mean±SD). c) Apoptotic effects assayed by PI/Annexin V-FITC dual staining and fluorescence-activated cell sorting analysis.

FIGS. 5a-5d. In vivo biodistribution and in vitro immune stimulation of RNA-MTX nanoparticles. FIG. 5a) Ex vivo fluorescent imaging of organs showing the distribution of RNA-MTX NPs in KB xenograft bearing mice. FIG. 5b) Ex vivo fluorescent imaging of organs showing the distribution of RNA-MTX NPs in KB xenograft bearing mice. (T: tumor, H: heart, S: spleen, Lu: lung, K: kidney, and Lv: liver). FIG. 5c) Native PAGE analysis of RNA-MTX samples and urine collected from mice within 1 hr of injection. FIG. 5d) In vitro immunogenicity of RNA-MTX NPs. LPS was used as a positive control (n=3, error bars are presented as mean±SD).

FIGS. 6A-6C. Improvement of water solubility of SN38 by conjugation to RNA strands. FIG. 6A) Schematic of conjugation of 6 SN38 to one RNA strand via click reaction. FIG. 6B) Gel electrophoresis and HPLC spectrum of RNA-6-ALK and RNA-6-SN38 strands. FIG. 6C) Solubility comparison of RNA-SN38 in water, SN38 in water, and SN38 in DMSO.

FIGS. 7A-7D. Assembly and characterization of motion element and motion element-SN38-E RNA nanoparticles. FIG. 7A) Schematic of functionalizing motion element RNA nanoparticles with 24 copies of SN38 and one copy of EpCAM RNA aptamer. FIG. 7B) Gel showing stepwise assembly of motion element-SN38-E and size comparison of motion element, motion element-SN38, and motion element-SN38-E. FIG. 7C) Size distribution of motion element and motion element-SN38-E. FIG. 7D) Thermostability of motion element and motion element-SN38-E demonstrated by annealing profile.

FIG. 8. In vitro cell binding and internalization of motion element-SN38-E RNA nanoparticles. Confocal images of HT29 cells after incubation with motion element-EpCAM and motion element-SN38-E, respectively. (Blue: nucleus; Green: Cytosol; Red: RNA nanoparticle)

FIGS. 9A-9B. In vitro cancer suppression study of motion element-SN38-E RNA nanoparticles. FIG. 9A) Cytotoxicity study by MTT assay in HT29 cells incubated with RNA nanoparticles and SN38 for 48, 72, and 96 h, respectively. FIG. 9B) In vitro apoptotic effects of motion element-SN38-EpCAM by PI/Annexin V-FITC dual staining and FACS analysis (Q2=Annexin V-FITC positive & PI positive, indicating late apoptotic and dead cells; Q3=Annexin V-FITC positive & PI negative, indicating early apoptotic cells).

FIGS. 10A-10C. In vitro immune response and in vivo tumor inhibition of motion element-SN38-E RNA nanoparticles. FIG. 10A) Evaluation of IFN-a and IL-6 production after incubating motion element-SN38-EpCAM with macrophage-like cells by ELISA. FIG. 10B) Intravenous treatment of nude mice bearing orthotopic HT29 xenografts with motion element-EpCAMapt and control groups every three days for a total of 5 injections (indicated by arrows). Mice tumor size was monitored during the time course of treatments (n=5 biologically independent animals, statistics was calculated by two-tailed unpaired t-test presented as mean±SEM, *p<0.05, **p<0.01, ***p<0.001). FIG. 10C) Tumor weight comparison.

FIGS. 11a-11b. Synthesis of DCL-N₃ and conjugation of trivalent DCL on RNA. FIG. 11a) Scheme showing the modification of DCL with azido group via NHS ester reaction. FIG. 11b) Reaction scheme showing the modification of trivalent DCL on RNA via copper-catalyzed click reaction.

FIGS. 12a-12e. Construction and characterization of PSMA-targeting RNA nanoparticles. FIG. 12a) 16% urea PAGE analysis of synthesized RNA and RNA-DCL conjugates. FIG. 12b) Native PAGE analysis of the step-wise assembly of PSMA-targeting RNA nanoparticles. FIG. 12c) TGGE characterization d) Serum stability (n=3, error bars are presented as mean±SD). FIG. 12e) Hydrodynamic size of motion element-based nanoparticles.

FIG. 13. Flow cytometry analysis of different RNA nanoparticles binding to PC3 and PC3 PIP cells. Y axis indicates the cell counts while X axis suggests the fluorescence intensity.

FIG. 14. Flow cytometry analysis of different concentrations (50 nM and 100 nM) of RNA nanoparticles binding to PC3 and PC3 PIP cells. Y axis indicates the cell counts while X axis suggests the fluorescence intensity.

FIG. 15. Confocal microscopy imaging of cellular uptake of RNA nanoparticles to PC3 and PC3 PIP cells (blue: nuclei; green: cytoskeleton; red: RNA nanoparticles).

FIG. 16. Ex vivo fluorescent imaging of organs showing the distribution of RNA nanoparticles in PC3 PIP xenograft bearing mice (T: tumor, H: heart, S: spleen, Lu: lung, K: kidney, and Lv: liver).

FIG. 17a-17b. Radiolabeling of RNA-NOTA nanoparticles and PET imaging of prostate cancer in PC3 PIP xenograft bearing mice (CPM: counts per minute; RCP: radiochemical purity; Green arrows indicate the location of PC3 xenograft and the blue arrows indicate the bladder).

FIG. 18 illustrates embodiments of a therapeutic structure disclosed herein.

FIG. 19 illustrates an embodiment of a therapeutic structure containing a motion element, an MTX and a CPT for cancer therapy.

FIG. 20 illustrates an embodiment of a therapeutic structure containing an MTX and a motion element to deliver multiple chemical drug molecules PTX to cancer in vivo using a positive cancer tissue targeting mechanism.

FIG. 21 illustrates an embodiment of a therapeutic structure containing an MTX and a motion element to deliver multiple chemical drug molecules PTOX to cancer in vivo using a positive cancer tissue targeting mechanism.

FIG. 22 illustrates an embodiment of a therapeutic structure containing an MTX and a motion element to deliver multiple chemical drug molecules SN38 to cancer in vivo using a positive cancer tissue targeting mechanism.

FIG. 23 illustrates an embodiment of a therapeutic structure containing an MTX and a motion element to deliver multiple chemical drug molecules BMS001 to cancer in vivo using a positive cancer tissue targeting mechanism.

FIG. 24 illustrates an embodiment of a therapeutic structure containing an MTX and a motion element to deliver multiple chemical drug molecules BMS8 to cancer in vivo using a positive cancer tissue targeting mechanism.

FIG. 25 illustrates an embodiment of a therapeutic structure containing an a MTX and a motion element to deliver multiple chemical drug molecules BMS37 to cancer in vivo using a positive cancer tissue targeting mechanism.

FIG. 26 illustrates an embodiment of a therapeutic structure containing an MTX and a motion element to deliver multiple chemical drug molecules BMS242 to cancer in vivo using a positive cancer tissue targeting mechanism.

FIG. 27 illustrates an embodiment of a therapeutic structure containing an MTX and a motion element to deliver multiple chemical drug molecules LY294002 to cancer in vivo using a positive cancer tissue targeting mechanism.

FIG. 28 illustrates an embodiment of a therapeutic structure containing an MTX and a motion element to deliver multiple chemical drug molecules PI3K-IN-10 to cancer in vivo using a positive cancer tissue targeting mechanism.

FIG. 29 illustrates an embodiment of a therapeutic structure containing an MTX and a motion element to deliver multiple chemical drug molecules MTX to cancer in vivo using a positive cancer tissue targeting mechanism.

FIG. 30 illustrates an embodiment of a therapeutic structure containing a DCL and a motion element to deliver multiple chemical drug molecules CPT to cancer in vivo using a positive cancer tissue targeting mechanism.

FIG. 31 illustrates an embodiment of a therapeutic structure containing a DCL and a motion element to deliver multiple chemical drug molecules PTX to cancer in vivo using a positive cancer tissue targeting mechanism.

FIG. 32 illustrates an embodiment of a therapeutic structure containing a DCL and a motion element to deliver multiple chemical drug molecules PTOX to cancer in vivo using a positive cancer tissue targeting mechanism.

FIG. 33 illustrates an embodiment of a therapeutic structure containing a DCL and a motion element to deliver multiple chemical drug molecules SN38 to cancer in vivo using a positive cancer tissue targeting mechanism.

FIG. 34 illustrates an embodiment of a therapeutic structure containing a DCL and a motion element to deliver multiple chemical drug molecules BMS001 to cancer in vivo using a positive cancer tissue targeting mechanism.

FIG. 35 illustrates an embodiment of a therapeutic structure containing a DCL and a motion element to deliver multiple chemical drug molecules BMS8 to cancer in vivo using a positive cancer tissue targeting mechanism.

FIG. 36 illustrates an embodiment of a therapeutic structure containing a DCL and a motion element to deliver multiple chemical drug molecules BMS37 to cancer in vivo using a positive cancer tissue targeting mechanism.

FIG. 37 illustrates an embodiment of a therapeutic structure containing a DCL and a motion element to deliver multiple chemical drug molecules BMS242 to cancer in vivo using a positive cancer tissue targeting mechanism.

FIG. 38 illustrates an embodiment of a therapeutic structure containing a DCL and a motion element to deliver multiple chemical drug molecules LY294002 to cancer in vivo using a positive cancer tissue targeting mechanism.

FIG. 39 illustrates an embodiment of a therapeutic structure containing a DCL and a motion element to deliver multiple chemical drug molecules PI3K-IN-10 to cancer in vivo using a positive cancer tissue targeting mechanism.

FIG. 40 illustrates an embodiment of a therapeutic structure containing a DCL and a motion element to deliver multiple chemical drug molecules DCL to cancer in vivo using a positive cancer tissue targeting mechanism.

FIG. 41 illustrates an embodiment of a therapeutic structure containing a UAMC1110 and a motion element to deliver multiple chemical drug molecules CPT to cancer in vivo using a positive cancer tissue targeting mechanism.

FIG. 42 illustrates an embodiment of a therapeutic structure containing a UAMC1110 and a motion element to deliver multiple chemical drug molecules PTX to cancer in vivo using a positive cancer tissue targeting mechanism.

FIG. 43 illustrates an embodiment of a therapeutic structure containing a UAMC1110 and a motion element to deliver multiple chemical drug molecules PTOX to cancer in vivo using a positive cancer tissue targeting mechanism.

FIG. 44 illustrates an embodiment of a therapeutic structure containing a UAMC1110 and a motion element to deliver multiple chemical drug molecules SN38 to cancer in vivo using a positive cancer tissue targeting mechanism.

FIG. 45 illustrates an embodiment of a therapeutic structure containing a UAMC1110 and a motion element to deliver multiple chemical drug molecules LY294002 to cancer in vivo using a positive cancer tissue targeting mechanism.

FIG. 46 illustrates an embodiment of a therapeutic structure containing a UAMC1110 and a motion element to deliver multiple chemical drug molecules PI3K-IN-10 to cancer in vivo using a positive cancer tissue targeting mechanism.

FIG. 47 illustrates an embodiment of a therapeutic structure containing a UAMC1110 and a motion element to deliver multiple chemical drug molecules UAMC1110 to cancer in vivo using a positive cancer tissue targeting mechanism.

FIG. 48 shows cancer vasculature is rich in capillary blood vessels. Crosslinked agarose was used to test the motile property of RNA complex with different structures. 4WJ RNA has higher 37° C. diffusion in Agar than Fe or Au. Unpaired RNA has a low density but also displays a deformative and motile structure that enables rapid diffusion out of the well as evidenced by the lower concentration of nanoparticles around the well after 24 hours. RNA diffuses out of the matrix well at a higher rate and penetrates further out than Fe (Cy5) and Au (Cy5) nanoparticles indicating the unpaired RNA is able to diffuse faster than spherical denser particles.

FIG. 49 shows unpaired RNA diffuses out at a higher rate than dsRNA.

FIG. 50 shows unpaired RNA diffuses out faster than paired RNA; and RNA complexes move and diffused faster than the iron and gold.

FIGS. 51A and 51B show motile and deformable property of RNA square nanoparticles by optical tweezer. Optical tweezers were used to apply an extending force on the RNA nanoparticle, in which a deformation was seen (inlet), upon relaxation the RNA nanoparticle returned to its original shape matching the extension curve.

FIGS. 52A to 52C show the motile and deformable property of RNA nanoparticles allows for tunable construction and enhanced tumor accumulation. FIG. 52A shows the motile and deformable RNA nanoparticles are able to deform to slip through leaky blood vessels for high tumor accumulation. FIG. 52B shows controlled synthesis with defined size, shape, structure, and stoichiometry. Demonstration of stretching and motile and deformable of the pRNA-3WJ. FIG. 52C shows angles of original 3WJ stretched to accommodate different shapes of 2D or 3D RNA nanoparticles. In each of the structure, schematic and AFM/Cryo-EM images are shown.

FIGS. 53A and 53B shows rapid renal excretion of RNA nanoparticles appeared in mice urine after IV injection demonstrating the motile and deformable property. FIG. 53A shows urine excretion of 3WJ nanoparticles at different time point in gel. FIG. 53B shows AF647 Channel showing ds-, 3WJ- and 4WJ-RNA nanoparticles stability in urine. B-RNA in buffer before IV injection as control, U-RNA in urine. Renal excretion limit is 5.5 nm.

FIGS. 54A and 54B shows motile and deformable RNA nanoparticles result in stronger tumor retention. FIG. 54A shows organ 8 hr post-injection of various nanoparticles comparing tumor and organ accumulation (T:tumor, H:heart, S:spleen, Lu:lung, K: kidney, Li:liver). FIG. 54B shows quantification tumor:liver and tumor:kidney ratios.

FIG. 55 shows the motile and deformable property of RNA nanoparticles for body clearance, the indirect evidence of renal excretion. Longer circulation time with increasing nanoparticles size.

FIG. 56 shows the motile and deformable property of RNA complex demonstrated by comparing tumor and kidney retentions. Fluor-RNA 10 nm squares were imaged in kidney compared at 12 and 24 hr. Both squares excreted through kidney, while remaining in tumors.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Controlling the Multivalency of Methotrexate on RNA Nanoparticles for Optimized Cancer Targeting and Therapeutic Effect

Recent advances in RNA nanotechnology-based platforms demonstrated the potentials of using RNA as building material for in vivo therapeutic delivery (Pi, F, et al. Nat Nanotechnol 2018 13: 82-89; Piao, X, et al. Adv Sci (Weinh) 2019 6:1900951; Guo, S, et al. Nat Commun 2020 11:972). Owing to the highly programmable and controllable properties, various RNA nanoparticles have been developed as drug delivery systems (Khisamutdinov, E F, et al. Adv Mater 2016 28:10079-10087; Li, H, et al. Adv Mater 2016 28:7501-7507). Particularly, the motion element (Shu, D, et al. Nat Nanotechnol 6:658-667) originated from the packaging RNA (pRNA) of the phi29 DNA packaging motor emerged as a powerful tool to construct structurally defined RNA nanoparticles, such as triangles, squares (Khisamutdinov, E F, et al. Nucleic Acids Res 2014 42:9996-10004) and three-dimensional nanostructures including tetrahedrons and pyramids (Li, H, et al. Adv Mater 2016 28:7501-7507; Xu, C C, et al. Nano Research 2019 12:41-48). Previous studies have shown that the size and hydrophobic modification of RNA nanoparticles had a close tie with the in vivo biodistribution and circulation of nanoparticles (Jasinski, D L, et al. Hum Gene Ther 2018 29:77-86). For instance, an RNA square with a smaller sizer (5 nm) was quickly cleared from the mice through renal filtration, while its 20 nm counterpart exhibited significant retention in the liver (Jasinski, D L, et al. Mol Ther 2018 26:784-792). When the motion element motif was conjugated with molecules of different hydrophobicity, the distinct biodistribution profile was implicated by the molecular characteristics of nanoparticles. Therefore, these studies provided nanoparticle designers with some useful hints on tuning the physicochemical properties for optimum in vivo behavior. Although it is clear that nanoparticle size and shape are key factors in determining tumor uptake, current strategies also involve the conjugation of biofunctional moieties to nanoparticles for active targeting (Curk, T, et al. Proc Natl Acad Sci USA 2017 114:7210-7215). Therefore, the valency of ligands on nanoparticles may also contribute to the different in vivo biodistribution profiles. With this in regard, whether implementing multivalent targeting strategy on RNA nanoparticles will lead to enhanced in vivo tumor targeting has not been studied and therefore not fully understood.

Methotrexate (MTX), structurally similar to folate, can function as a targeting ligand to folate receptor (FR) as well as a therapeutic agent (Li, Y, et al. ACS Appl Mater Interfaces 2017 9:34650-34665). Its therapeutic effect is attributed primarily to inhibition of human dihydrofolate reductase (DHFR), an enzyme localized in the cytoplasm (Junyaprasert, V B, et al. Colloids Surf B Biointerfaces 2015 136:383-393). Recent experimental and theoretical studies have suggested that using multivalent low-affinity ligands can benefit selective cell targeting (Chittasupho, C. Ther Deliv 2012 3:1171-1187). Consequently, MTX, with a weaker binding to FR, presents itself as an ideal bi-functional molecule to be used in the multivalent targeting and inhibition of FR-overexpressed tumor in a controllable manner (Hao, F, et al. Pharmaceutics 11).

Here we present a strategy to modulate the ligand valency on motion element based RNA nanoparticles using MTX as a targeting and therapeutic agent. By varying the copies of functional groups on motion element scaffold, we made a series of motion element-MTX conjugates with different loading capacity, ranging from 1, 3, 18 to 28 copies. Physicochemical characterization of these RNA nanoparticles was performed to evaluate the hydrodynamic size, zeta potential, thermodynamic stability as well serum stability. More importantly, the serum protein binding profile of RNA-MTX conjugates was compared. Results have indicated a trend of increased serum protein binding with an increased number of MTX on nanoparticles. Further flow cytometry analysis and confocal microscope imaging results suggested the enhanced specific binding to folate receptor overexpressed in cancer cells in vitro as the MTX valency increases. Although the difference between 18 and 28 copies of MTX was not very significant in cellular uptake and inhibition, the in vivo cancer targeting and urinary excretion results implicated the effect of multivalent ligand binding. Taking advantage of the modular assembly and programmable valency, the nano-bio interfacial interaction can be well controlled, thus achieving optimum in vivo cancer targeting and therapy. This study demonstrated the importance of controllable multi-valent targeting in ligand-conjugated nanoparticle design and will provide rationale for proper design of RNA nanodelivery system in cancer treatment.

Results

Methotrexate-Azido Modification, Purification, and Characterization

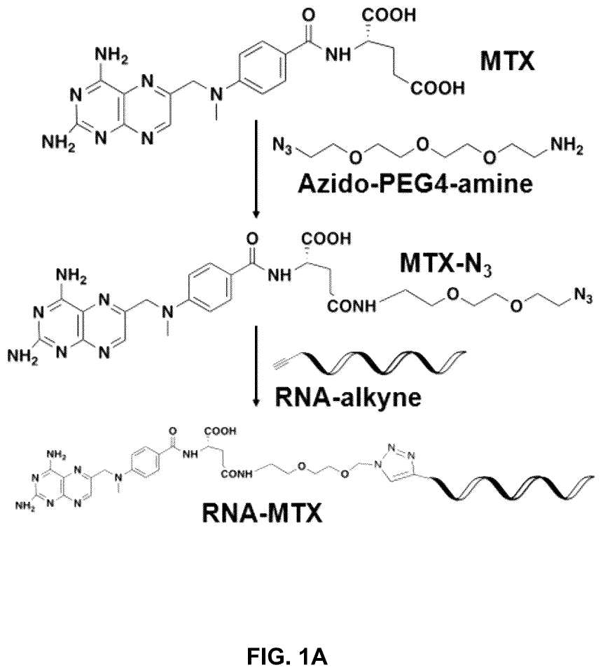

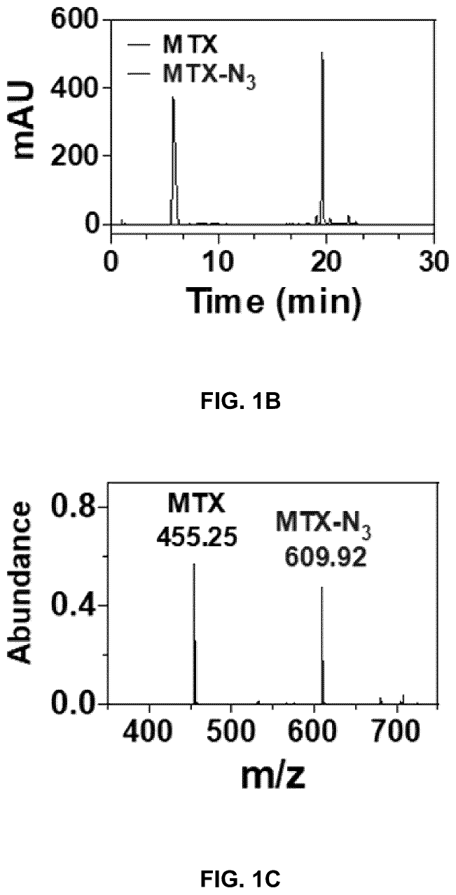

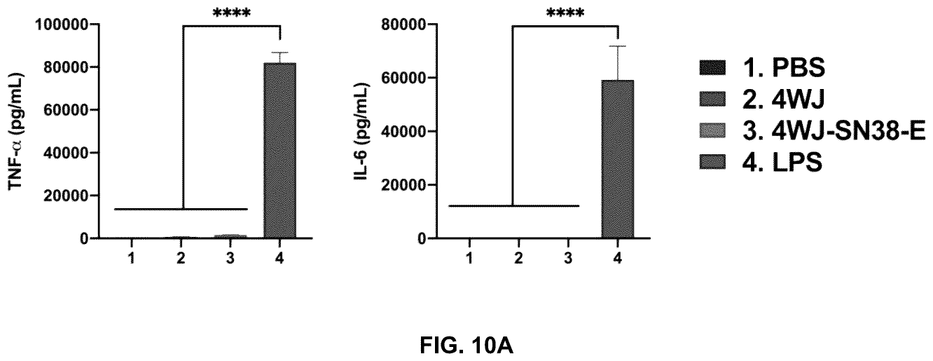

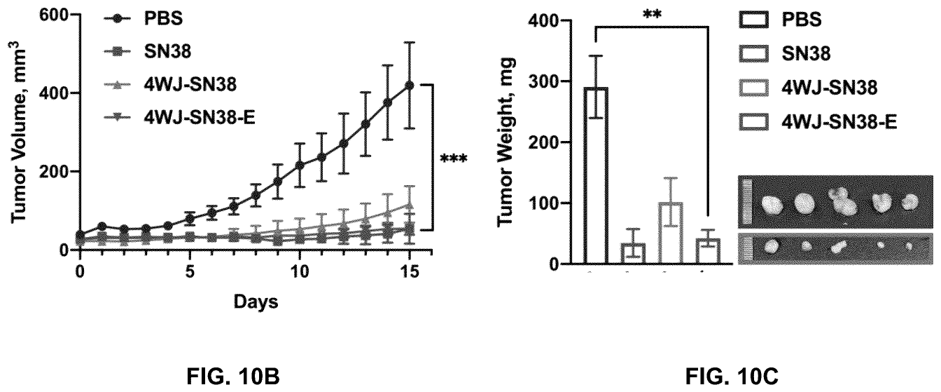

MTX was used as a bi-functional molecule in this study for cancer targeting and inhibition. To achieve multivalent conjugation, we first modified MTX with an azido group via EDC coupling reaction (FIG. 1A). The bi-functional linker, azido-PEG4-amine, was used to covalently form an amide bond with the carboxylic acid group on MTX and introduce an azido group for following click reaction with RNA. As a result of azido modification, the less polar MTX-N₃ compound was purified by reverse-phase HPLC. The purified compound was analyzed on a C18 reverse-phase column with comparison to MTX as shown in FIG. 1B. A major peak was observed at a longer retention time (approximately 19 min) for MTX-N₃. While the raw compound MTX was eluted out of the column at about 6 min. Further mass spectrometry analysis of the MTX and purified MTX-N₃ (FIG. 10) showed an m/z value of 455.25 and 609.92, respectively, which was attributed to the [M+H]⁺. These results confirmed the single covalent attachment of azido group on MTX with a PEG chain linker.

Construction of an RNA Nanoparticle with Controllable Valency of Methotrexate

As shown in the reaction scheme (FIG. 1A), the conjugation of MTX-azido on RNA was accomplished by simple copper(I)-catalyzed azide-alkyne cycloaddition (CuAAC) reaction. To increase the loading capacity of MTX on RNA, internal and terminal alkyne groups were incorporated. Single terminal modification of RNA with alkyne group could be achieved using 5′-hexynyl phosphoramidite in the process of solid-phase RNA synthesis. Similarly, a trebler phosphoramidite was used to create a branch at the 5′-end of RNA for trivalent alkyne modification. To increase the flexibility of the conjugated ligand, four Uracil nucleotides were incorporated as a spacer after trivalent branching point. For internal modification of RNA, 2′-propargyl rC and rU amidites were used to introduce internal alkyne groups on RNA strands for MTX conjugation (See detailed sequence information in Materials and Method section). All other pyrimidines (C&U) were modified with 2′-fluorine (2′-F) to confer enzymatic stability on RNA.

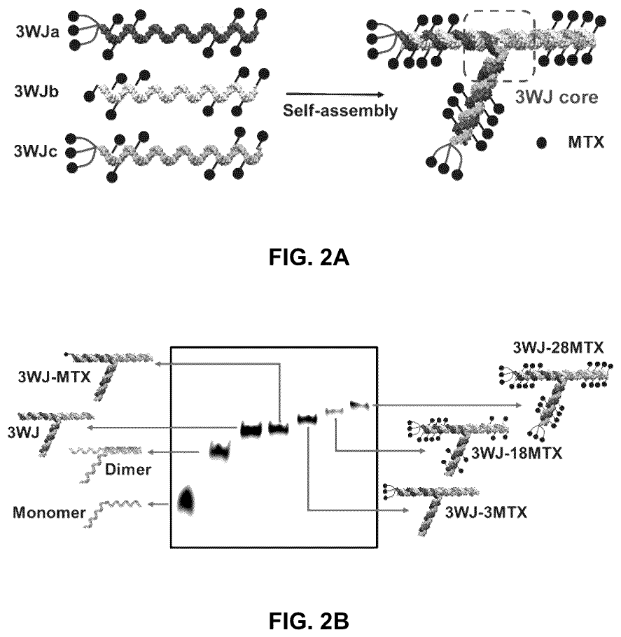

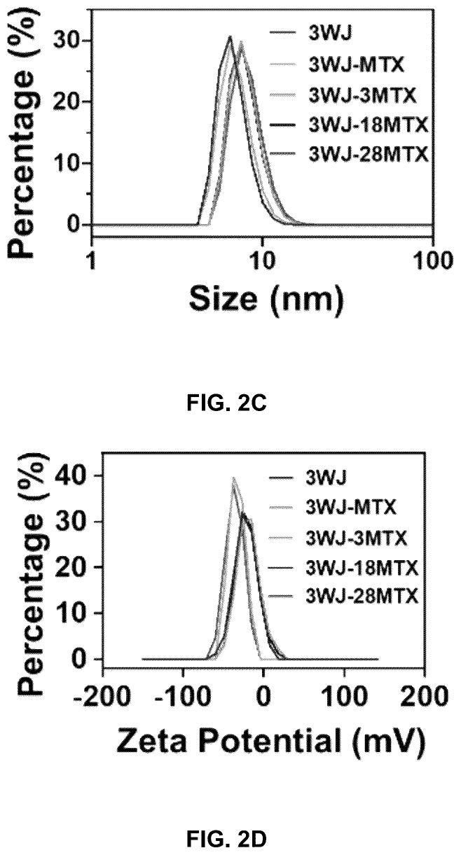

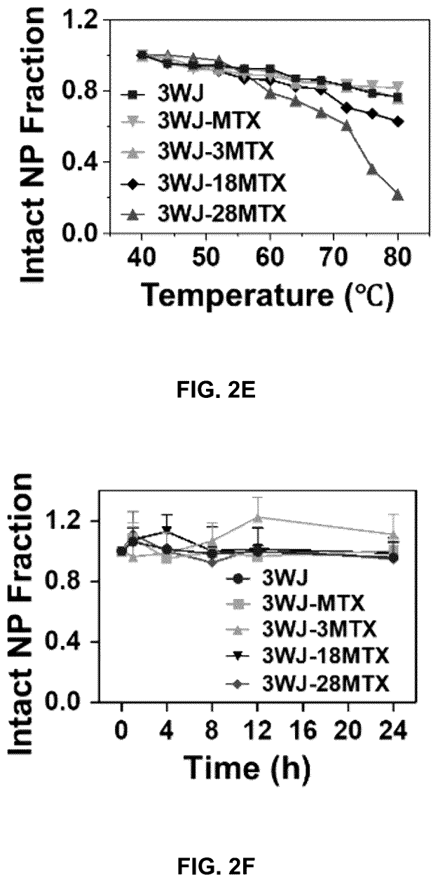

However, chemical conjugation on RNA strands may interfere with base pairing thus disrupting nanoparticle assembly. To address this concern, we utilized a new thermodynamically stable three-way junction (motion element) scaffold re-engineered from naturally occurring SF5 packaging RNA motif (Hao, Y, et al. RNA 2014 20:1759-1774) composed of three oligomers (a, b, and c). Previous studies suggested that the chemical modification on the core sequences impacted the thermodynamic stability of the nanostructure, which may lead to dissociation at a physiological temperature condition (Guo, S, et al. Nat Commun 2020 11:972). To ensure the loading capacity, the motion element core were extended with 11 base pairing (bp) on each helix (H1, H2, and H3) for MTX incorporation. The motion element core, exhibited unusually high thermodynamic stability. By modifying the single-stranded RNA with varying number of alkyne groups, different loading capacity of MTX ranging from 1, 3, 8, to 10 on each RNA strand was achieved. For instance, when motion elementa-10MTX, motion elementb-8MTX and motion elementc-10MTX were assembled, a total of 28 MTX molecules were incorporated into a motion element complex as shown in FIG. 2A. Therefore, assembly of motion element constructs using three separate MTX conjugated RNA strands made it possible to control the valency of MTX with ease. To construct motion element nanoparticles, the composite strands motion elementa, motion elementb and motion elementc were mixed with 1:1:1 stoichiometry ratio in PBS buffer followed by the annealing process.

Characterization of RNA nanoparticles with MTX As three individual RNA oligos were incorporated into one nanoparticle complex, we performed a stepwise assembly characterization by 10% native polyacrylamide gel electrophoresis (PAGE). As shown in the FIG. 2B, gel mobility shift was observed from monomer, dimer to motion element trimer complex. Assemblies of motion element-MTX, motion element-3MTX, motion element-18MTX and motion element-28MTX nanoparticles were observed with slower gel migration, indicating the successful formation of motion element-based structures. As the physicochemical properties of RNA nanoparticles play critical roles in governing the in vivo behavior, we did further characterizations on these RNA nanoparticles regarding size, shape and thermostability.

Studies have shown that nanoparticles with 6 nm or smaller diameter were able to be eliminated from the body by renal clearance, while larger nanoparticles exhibited more significant liver accumulation (Jasinski, D L, et al. Mol Ther 2018 26:784-792; Du, B, et al. Nature Reviews Materials 2018 3:358-374). Dynamic size scattering (DLS) analysis was performed to measure the hydrodynamic size of particles of all RNA-MTX nanoparticles. The results showed a similar size range of all RNA-MTX nanoparticles (FIG. 2C). Slight increase in diameter was observed from motion element (7.9±1.7 nm) to motion element-MTX (9.1±1.8 nm) as a result of 5′-terminus modification. Due to trivalent MTX modification, motion element-3MTX, motion element-18MTX and motion element-28MTX were measured with a diameter at around 10 nm (Table 1) (Size was presented as mean±SD of one size distribution). As the surface potential of nanoparticles may interfere with cellular interaction by electrostatic force, the zeta potentials of RNA-MTX were measured as shown in FIG. 2D. Summarized data in Table 1 indicated the negative charge of all RNA complexes even after multiple MTX conjugation, which reduces the non-specific interaction with negatively-charged cell membranes.

TABLE 1
Summary of physicochemical characterization
results of all RNA NPs.

| RNA NP | Size (nm) | Zeta Potential (mV) | Tₘ (° C.) |
|---|---|---|---|
| motion element | 7.9 ± 1.7 | −23.8 ± 13.5 | >80 |
| motion element-MTX | 9.1 ± 1.8 | −26.9 ± 11.0 | >80 |
| motion element-3MTX | 10.2 ± 2.0 | −24.5 ± 11.7 | >80 |
| motion element-18MTX | 11.0 ± 2.1 | −24.2 ± 11.2 | >80 |
| motion element-28MTX | 11.2 ± 1.6 | −27.8 ± 9.7 | 72 |

Other key factors to consider in RNA nanoparticle construction include thermodynamic stability and serum stability. Higher drug loading or ligand conjugation capacity may be detrimental to the base pairing and folding of RNA, potentially resulting in the disassembly of the nanostructure (Piao, X, et al. Adv Sci (Weinh) 2019 6:1900951). Therefore, we evaluated the melting temperature (Tₘ) of RNA-MTX complex by temperature-gradient gel electrophoresis (TGGE) (Benkato, K, et al. Methods Mol Biol 2017 1632:123-133). With majority of the constructed nanoparticles remaining intact at a temperature over 80° C., motion element, motion element-MTX, motion element-3MTX, and motion element-18MTX showed exceptional stability against high temperature. motion element-28MTX was found to be less stable as a result of high loading of conjugated MTX molecules (FIG. 2E). However, the estimated Tₘ at 72° C. was still much higher than the physiological temperature, preventing the disassembly of the nanostructure under in vivo systemic circulation. In addition, we conducted serum stability assays to compare the chemical stability of various RNA-MTX nanoparticles. Incubation with 10% FBS up to 24 hrs did not cause obvious degradation of RNA-MTX nanoparticles regardless of the valency of MTX, demonstrating that the MTX conjugation did not alter the serum stability of RNA nanostructures.

Serum Protein Binding Impacts the Specific Binding

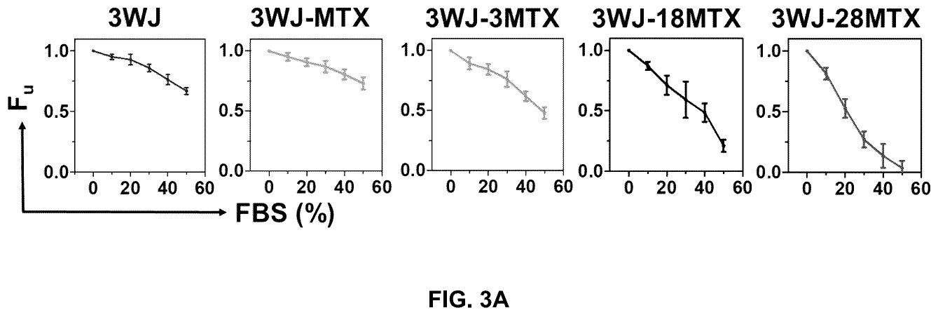

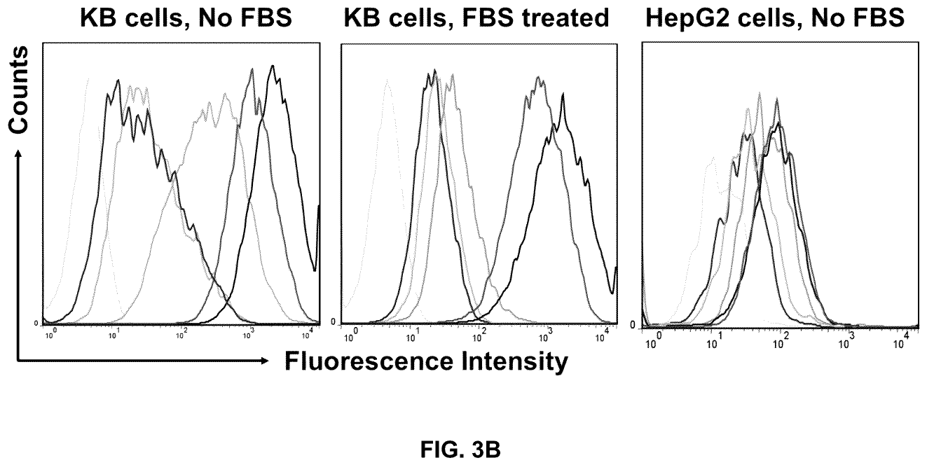

It was hypothesized that an increased ligand display on the surface of nanoparticles will contribute to stronger binding to target cells in vitro and tumor tissue in vivo. However, upon introduction into biological fluids, the nanoparticles encounter a complex biological environment and bind to serum proteins within a very short period of time (Chen, F F, et al. Nature Nanotechnology 2017 12:387-393; Tenzer, S, et al. Nature Nanotechnology 2013 8:772-U1000). The serum proteins form a “protein corona” on the nanoparticle, facilitating the liver uptake and metabolism. Moreover, the “protein corona” formation may impact the renal clearance of nanosized particles as a result of overall size increase (Bertrand, N, et al. Nat Commun 2017 8:777; Lundqvist, M, et al. ACS Nano 2011 5:7503-7509). Therefore, we took a closer look at the serum protein binding of RNA-MTX nanoparticles. By incubating with a gradient concentration of FBS serum, the RNA nanoparticles were analyzed by gel electrophoresis to show the unbound fraction in the mixture. Quantification results plotted as FIG. 3A elucidated the unbound fraction of total (Fᵤ) as a function of increasing FBS concentration. All the RNA nanoparticles showed a trend of decreasing F, when the FBS concentration increased from 0 to 50%. Intriguingly, as more MTX ligands were anchored onto the motion element nanoparticle, more substantial serum protein binding was observed. It is possible that the surface characteristic of RNA nanoparticles shifted to be less polar as a higher density of MTX ligand was presented. At a concentration 50% FBS, the majority of motion element-18MTX and motion element-28MTX were bound with the serum protein while less was found for motion element, motion element-MTX and motion element-3MTX. Previous reports suggested that the serum protein binding on a nanoparticle with targeting ligands may disable its targeting capability due to the shielding of conjugated biofunctional moieties (Salvati, A, et al. Nat Nanotechnol 2013 8:137-143). In this study, we aimed to increase the valency of MTX ligands for enhanced targeting, which, however, also resulted in an increased serum protein binding. It remained unclear that whether higher valency of MTX will necessarily lead to stronger cellular targeting and tumor targeting, as complicated by the increased serum protein binding. Therefore, we conducted the flow cytometry analysis to evaluate the RNA-MTX complex binding to FR overexpressed KB cells under different conditions. Without the pre-incubation with FBS, the specific binding to KB cells was gradually enhanced from motion element, motion element-MTX, to motion element-18MTX (FIG. 3B). It was interesting to find that motion element-28MTX showed less binding to KB cells compared to motion element-18MTX. To evaluate whether the FBS binding to RNA-MTX nanoparticles will impact the in vitro cellular targeting, we treated the RNA nanoparticles with 50% FBS before incubation with KB cells. Flow cytometry analysis results suggested a distinct decrease of cellular binding in motion element-3MTX group while other groups retained similar binding (FIG. 3B, middle figure). With comparison of the fluorescent shift, motion element-MTX remained low binding to target KB cells regardless of FBS pre-treatment. motion element-18MTX and motion element-28MTX exhibited less binding to KB cells after FBS pre-treatment which was possibly aided by the shielding of partial MTX ligands anchored on the nanoparticles. As for the motion element-3MTX, its targeting capability was significantly weakened by FBS pre-treatment, demonstrating the shielding of MTX by “protein corona”. In another control group using HepG2 cell as a FR-negative control cell line, the enhanced binding to cells using MTX as targeting ligands was not very significant (FIG. 3C). Therefore, we demonstrated that the multivalent targeting ligand strategy using MTX implemented on motion element was able to retain the targeting capability without being impacted by the protein binding.

Internalization of RNA-MTX into KB Cells

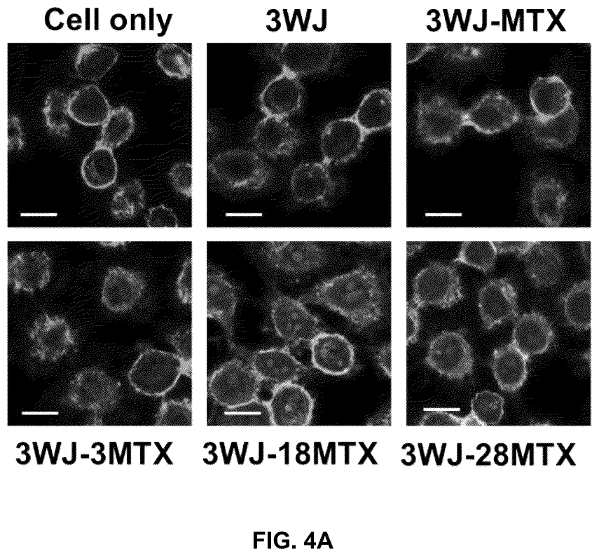

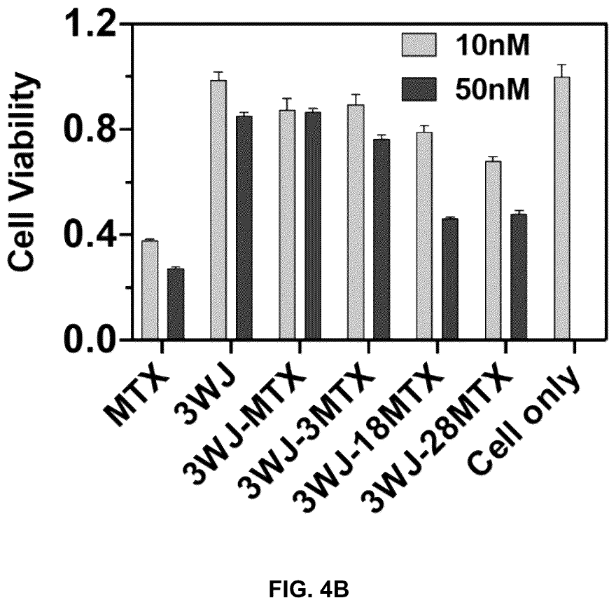

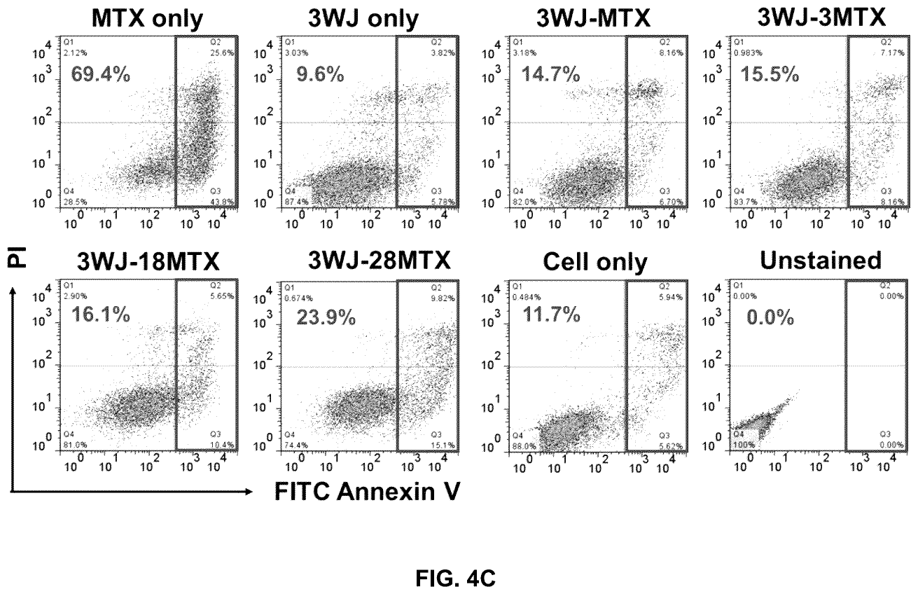

To evaluate whether the conjugated MTX ligand would facilitate the internalization of RNA nanoparticles, we carried out cell internalization assay by confocal microscopy imaging. As shown in FIG. 4A, motion element-18MTX and motion element-28MTX exhibited substantial cell internalization. In contrast, RNA nanoparticles conjugated with less or without MTX ligand did not show obvious cellular uptake. Confocal images scanned under three separate emission channels clearly showed the difference in cellular uptake among all RNA-MTX groups. As the results suggested a strong correlation with MTX valency, it's arguably evidenced that an increased valency facilitated the receptor-mediated cellular uptake in vitro.

In Vitro Inhibition Effect of RNA-MTX Complex

In the present study, MTX primarily functions as a targeting ligand and therapeutic agent. Here, we evaluated the therapeutic effect of all RNA-MTX to obtain more information on the in vitro inhibition. MTT (3-(4,5-dimethylthiazol-2-yl)-2,5-diphenyltetrazolium bromide) assay was performed at two different doses (10 nM and 50 nM) of RNA nanoparticles. As suggested in FIG. 4B, higher dose (50 nM) induced more inhibition compared to lower dose among all test groups. More significant inhibition was seen in motion element-18MTX and motion element-28MTX groups with comparable performance at a 50 nM concentration, whereas mono- and tri-valent conjugation of MTX did not induce noticeable cytotoxicity. Though the cell viability remained higher than MTX control group, it's believed that the MTX payload on motion element nanoparticles needs longer time to be released for therapeutic function. Different mechanisms of cellular uptake of RNA-MTX complex and MTX could also lead to the delayed inhibition.

As an antimetabolic drug, MTX can successfully deactivate cell metabolism through apoptosis (AlBasher, G, et al. Onco Targets Ther 2019 12:21-30). We further evaluated the apoptotic effects induced by RNA-MTX nanoparticles in FR-overexpressed KB cells. The Propidium iodide (PI) and FITC Annexin V double staining analysis also confirmed the apoptosis was induced by RNA-MTX nanoparticles. The results showed a more significant apoptotic effect was induced as the valency of MTX on motion element increased (FIG. 4C). It was determined that 16.1% and 23.9% of cells underwent apoptosis after being treated with motion element-18MTX and motion element-28MTX, respectively. motion element, motion element-MTX and motion element-3MTX were less effective in inducing cell apoptosis, whereas the positive control MTX group showed 69.4% apoptosis.

Controlling the In Vivo Biodistribution of RNA-MTX by Modulating the Valency of MTX on RNA Nanoparticles

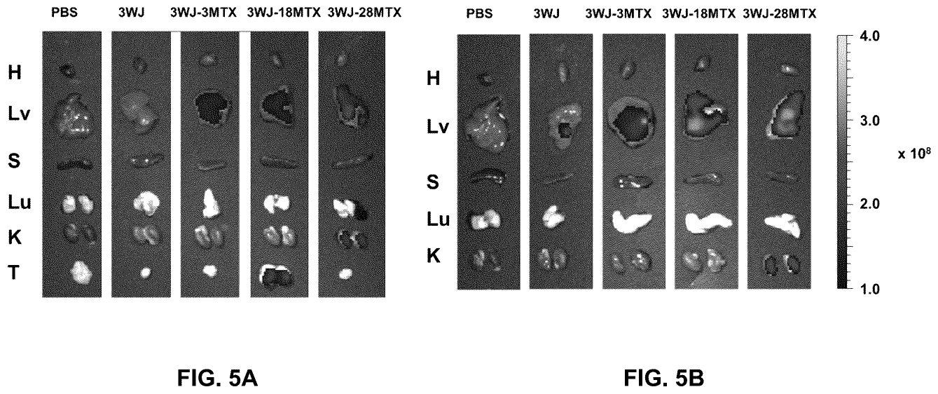

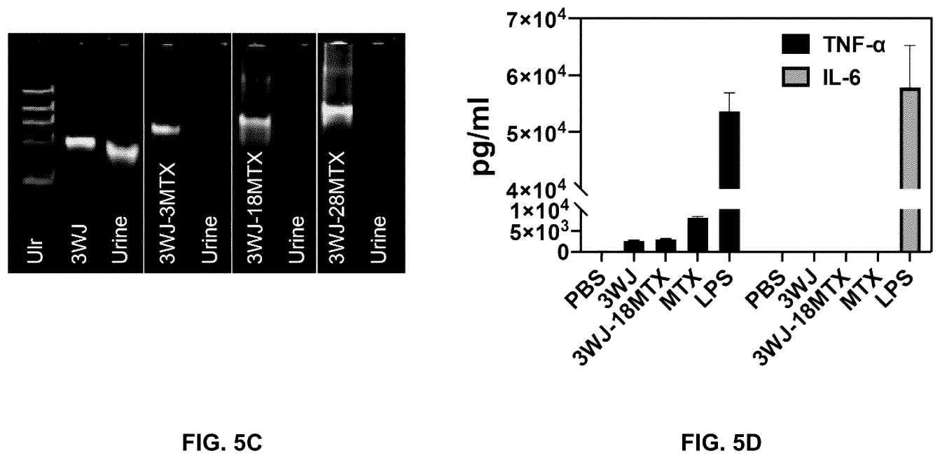

The MTT and PI/Annexin-V assay confirmed the therapeutic effect of RNA-MTX nanoparticles in vitro. However, the in vivo drug delivery was more complicated than an in vitro scenario where the cells were incubated directly with therapeutics. When the nanoparticles were introduced in vivo, biological barriers such as mononuclear phagocyte system (MPS) pose a challenge for successful cancer targeting (Blanco, E, et al. Nat Biotechnol 2015 33:941-951). For instance, rapid “protein corona” formation facilitate the liver accumulation, reducing the blood circulation of nanoparticles (Kim, S M, et al. J Control Release 2017 267:15-30). Moreover, fast renal clearance of smaller nanoparticles causes a short half-life of injected nanoparticles, contributing to a low bioavailability. As such, controlling the properties of nanoparticles for elongated blood circulation and enhanced tumor targeting is of pivotal significance. Our strategy in tuning the valency of MTX in the present study offers a new idea to achieve this goal. To compare the in vivo targeting effect and organ biodistribution, we performed fluorescent ex vivo organ imaging after systemic administration of fluorescently labeled RNA nanoparticles in mice. Eight hours post-injection, vital organs and tumor were taken out from mice for imaging. In the KB xenograft-bearing nude mice, organ images showed an increasing liver accumulation when the MTX valency increased (FIG. 5A). Interestingly to find that the motion element-18MTX exhibited highest tumor uptake while other groups did not show obvious fluorescent signal in tumor tissue. In addition, we observed the fluorescent signal was in the kidney exclusively in motion element-28MTX experiment group, indicating a longer clearance time for motion element-28MTX nanoparticle. For the motion element-3MTX group, the serum protein binding on the surface made the trivalent ligands inaccessible to the folate receptor, thereby preventing the active targeting to tumor. In another in vivo biodistribution study, a similar trend in liver accumulation and kidney filtration was seen in mice without tumor xenograft (FIG. 5B). It's very likely that the strongest serum protein absorbance on motion element-28MTX among all groups resulted in significant liver accumulation, and the increased size after “protein corona” formation prevented fast kidney filtration. As the renal clearance usually occurs within a short period of post-injection time, we collected the urine of mice in the first 1 h after administration of RNA nanoparticles. Native PAGE analysis of the urine sample suggested the rapid renal clearance of intact motion element nanoparticles (FIG. 5C), whereas other RNA-MTX complexes did not shown urinary secretion within 1 hr. Taken together, we believed that the valency of MTX on motion element RNA nanoparticles played a critical role in determining the distribution, serum protein binding, liver accumulation, tumor targeting as well as renal clearance. By modulating the number of MTX per motion element nanoparticle, optimum biodistribution and cancer targeting effects can be achieved.

Undetectable Immune Stimulation In Vitro

Immunological evaluations are one of the most important drug evaluations prior to their translation into the practical use (Dwivedi, P D, et al. J Biomed Nanotechnol 2011 7:193-194). Our recent publications suggested that the immunogenicity of RNA nanoparticles is size, shape, and sequence dependent (Guo, S, et al. Mol Ther Nucleic Acids 2017 9:399-408). As a biocompatible material, RNA has been developed as FDA-approved therapeutic agent for gene therapy (Adams, D, et al. N Engl J Med 2018 379:11-21). Previous studies have shown that MTX may reduce immune surveillance as a result of decreased immunoglobulin production and cellular immune response (Bedoui, Y, et al. Int J Mol Sci 2019 20). However, after conjugating to RNA nanoparticles, the immunostimulatory effect of RNA-MTX may be altered. Therefore, we performed an in vitro TNF-a and IL-6 induction in RAW264.7 cells as shown in FIG. 5D. The results showed undetectable or negligible tumor necrosis factor α (TNF-α) and interleukin 6 (IL-6) induction compared to the positive control lipopolysaccharides (LPS), demonstrating the safety profile of RNA-MTX nanoparticles regarding immunogenicity.

### CONCLUSIONS

When a biological environment interacts with introduced nanomedicine, in a complicated manner where the physicochemical properties of nanoparticles play a critical role; whether an increased valency of targeting ligands on RNA nanoparticles leads to better in vivo behavior remains elusive. To answer this question, we investigated the effect of ligand valency on the biodistribution profile and cancer targeting performance of RNA nanoparticles. By using MTX as a bifunctional molecule, we manipulated the valency of MTX on motion element scaffolds. In vitro cancer targeting and therapeutic effects demonstrated the controllable binding affinity and inhibition effect to target cells. As for the therapeutic efficacy, it was reasonable to believe that a higher drug loading on the nanocarrier would achieve a higher drug delivery efficiency. However, a chemical drug conjugation could alter the surface characteristics of RNA nanoparticles as a result of introducing more hydrophobic moieties, which may cause solubility issues as well as non-specific interaction with cell membranes. Additionally, the release of therapeutic agents from a nanocarrier intracellularly involves a complicated process where the covalent attachment may be cleaved, or the delivery vehicle may be decomposed. Thus, the covalently conjugated MTX may not be fully released from RNA nanoparticles if the payload continues to increase.

Serum protein binding profiles of different RNA-MTX nanoparticles were impacted by the valency of MTX as well, which consequently contributed to a different in vivo cancer targeting effect, renal clearance rate and liver accumulation. Ex vivo fluorescent organ imaging indicated the superior cancer targeting of motion element-18MTX nanoparticles and the most significant liver accumulation of motion element-28MTX nanoparticles. Urinary analysis suggested the fast clearance of motion element nanoparticles which showed the least serum protein binding. Taken together, we believe that the valency of targeting ligand will also be a key consideration in the nanomedicine design to control the in vivo fate and this rationale provides useful information for multivalent ligand design.

Methods and Materials

(S)-(+)-Camptothecin, 4-Dimethylaminopyridine (DMAP), Copper(I) Bromide (CuBr), Tris[(1-benzyl-1H-1,2,3-triazol-4-yl)methyl]amine (TBTA) were purchased from Sigma-Aldrich (St. Louis, MO). 1-Ethyl-3-(3-dimethylaminopropyl)carbodiimide hydrochloride (EDC) was obtained from Thermo Fisher Scientific (Rockford, IL). 6-Azidohexonic acid was ordered from Chem-Impex International, Inc (Wood Dale, IL). All solvents for organic synthesis were supplied by Sigma-Aldrich (St. Louis, MO). Chemicals, solvents and other supplies for solid-phase oligonucleotide synthesis and HPLC were purchased from Bioautomation (Irving, TX) and Glen Research (Sterling, VA). RNA phosphoramidites (2′-TBDMS bz-A-CE, 2′-TBDMS ibu-G-CE, 2′-F U-CE and 2′-F ac-C-CE) were obtained from Bioautomation Corp. Functional modification amidites were purchased from ChemGenes Corp. Chemical reagents for RNA synthesizer were purchased from Sigma-aldrich unless otherwise stated. AFDye 647 NHS ester was obtained from Fluoroprobes. HPLC-grade water and acetonitrile were purchased from Thermo Fisher. Other reagents are purified by the general methods or used as received.

Design of RNA Motion Element Nanoparticles with Multiple Methotrexate Conjugation

For multiple MTX conjugation, the RNA oligos were synthesized with multiple alkyne groups followed by reaction with azide-modified methotrexate. As the drug conjugation may interfere with the base pairing, the alkyne modification at central motif of motion element was avoided.

Synthesis, Purification, and Characterization of Methotrexate-Azido

Methotrexate (30 mg) was dissolved in DMSO. 1-Ethyl-3-(3-dimethylaminopropyl) carbodiimide (EDC) (25 mg) was activated in MES buffer followed by mixing with methotrexate and N-Hydroxysuccinimide (NHS) (25 mg). After stirring the solution for 20 min, azide-PEG4-amine in 0.1 M sodium bicarbonate was added into the solution. The reaction mixture was kept stirring overnight at RT. Reverse-phase HPLC with Zorbax C18 column was used for purification of MTX-azido. A gradient of 2%-98% of acetonitrile was applied over 40 min. Absorbance at 305 nm was recorded. Purified MTX-azide was characterized by mass spectrometry using Thermo LTQ Orbitrap.

Solid-Phase Synthesis of RNA

RNA oligonucleotides with multiple alkyne modification were synthesized via standard solid-phase RNA synthesis on Biosset ASM-800 DNA synthesizer using 1000 Å universal CPG column. 2′-fluoro modified Cytidine and Uridine phosphoramidites were used for all pyrimidine modification on RNA. 5′-terminnal and internal alkyne groups were modified using 5′-hexynyl phosphoramidites (Glen Res. Corp.) and 2′-propargyl phosphoramidites (ChemGenes Corp.) respectively. For trivalent alkyne modification of RNA, the trebler phosphoramidite (Glen Res. Corp.) was used followed by 5′-hexynyl modification. RNA oligomers were purified by desalting using Glen Pak purification cartridges (Glen Res. Corp). The RNA sequences (lower-case letters indicate 2′-F nucleotides; letters labeled with * indicate 2′-propargyl nucleotides; letter H indicate the 5′-hexynyl modification; letter T indicates the trebler modification) are:

SF5a-alkyne:
(SEQ ID NO: 1)
HccuAuucAGGuGcGuGcuGGuGcuAccGAuGuAAuucAA
SF5a-M3-alkyne:
(SEQ ID NO: 2)
HuuuuTccuAuucAGGuGcGuGcuGGuGcuAccGAuGuAAuucAA
SF5a-M10-alkyne:
(SEQ ID NO: 3)
HuuuuTccU*AU*uC*AGGuGcGuGcuGGuGcuAcC*GAU*GuAAU*u
C*AA
SF5 b-M8-alkyne:
(SEQ ID NO: 4)
HuU*GAAU*uAC*AucGGuAGcAcGGGcuGU*GC*GAGGCU*GAAC*AG
SF5 c-M10-alkyne:
(SEQ ID NO: 5)
HuuuuTcU*GU*uC*AGccucGcAcAGccAGcAC*GC*AC*CU*GAAU*
AGG

Methotrexate Conjugation on RNA

MTX-azide was conjugated to alkyne-modified RNA oligos via Copper-catalyzed azide-alkyne cycloaddition (CuAAC). A 100 mM CuBr solution (tBuOH:DMSO=1:3) was mixed with 100 mM TBTA (tBuOH:DMSO=1:3) in a ratio of 1:2 to yield Cu-TBTA complex. Next 10 μL of freshly prepared Cu-TBTA solution was added to 60 μL MTX-azide solution (DMSO, 20 mM) followed by adding 50 μL alkyne-modified RNA oligos solution (water, 1-2.5 mM measured by absorbance at 260 nm). The reaction was completed overnight at room temperature. Water was added to reach 400 μL (total volume) followed by adding 40 μL 3 M sodium acetate (pH 5.2) and 1100 μL ethanol. The tube was stored at −20° C. for at least two hours to precipitate RNA-MTX conjugate. After centrifugation at maximal speed (13100 RPM) at 4° C. for 30 minutes and washing the pellet with 70% cold ethanol, the pellet was dried by speed vacuum. Following this 100 μL water was used to dissolve RNA-MTX conjugate. RNA-MTX conjugate was further purified by reverse phase HPLC (Solvent A: 0.1 M TEAA (triethylamine acetate) in water and Solvent B: 75% acetonitrile and 25% water with 0.1 M TEAA).

Assembly and Characterization of RNA Nanoparticles

All RNA motion element samples were self-assembled by mixing three separate strands (motion elementa, motion elementb, and motion elementc) at equimolar concentration in PBS buffer. The “one-pot” assembly process was done by heating the mixture solution to 85° C. for 5 minutes and cooled it down to 4° C. at the rate of −2° C./min. The assembled complex was characterized by 12% native PAGE using TBE buffer (120V, 4° C., 90 minutes). All assembled RNA motion element samples studied in cell and animal experiments were filtered by 0.22 μm spin filter column. Filtered motion element samples were also used in the measurements of dynamic light scattering and zeta potential.

DLS and Zeta Potential Characterization of RNA Motion Element-M-MTX Samples

The apparent hydrodynamic diameter and zeta potential of the RNA nanoparticles in 1 μM in TMS buffer were determined using a Zetasizer Nano-ZS (Malvern Instrument) at 25° C. The laser wavelength was 633 nm. Results were plotted with data points using GraphPad Prism 8 software. Three independent runs with 8 measurements were performed and recorded.

Serum Stability Characterization of RNA Motion Element-M-MTX Samples

Assembled RNA nanoparticles were incubated in cell culture medium containing 10% (v/v) FBS at a final concentration of 1 μM at 37° C. After removal from 37° C. incubation after their respective time points, the samples were frozen in −80° C. to prevent any further degradation. The resulting samples were examined on 6% native PAGE followed by fluorescent imaging by Typhoon imaging system under ethidium bromide and Cy5 channel. Quantification analysis was performed using ImageJ software and plotted by GraphPad Prism 8 software.

Serum Binding Characterization of RNA Motion Element-M-MTX Nanoparticles

RNA nanoparticles at a concentration of 500 nM were incubated with FBS solution in 1×PBS buffer for 5 min at 37° C. A gradient final concentration of FBS (0%, 10%, 20%, 30%, 40%, and 50%) was used. An 8% native gel electrophoresis was used to characterize the RNA nanoparticles. Serum binding experiments for each particle were repeated three times, and error is indicated by error bars. To measure serum binding, Ethidium bromide (E.B.) signal from the RNA nanoparticle bands (lower bands) was integrated using ImageJ software. E.B. signal for each lane was normalized to the 0% serum band, and values were then plotted (the y axis is the fraction of nanoparticles unbound, and the x axis is the concentration of FBS used in incubation). The plot was generated by GraphPad Prism 8 software.

Thermostability of Motion Element-M-MTX Nanoparticles

Preassembled RNA nanoparticles were run in a 10% (w/v) native PAGE in TBE buffer at 100 V for 10 min at room temperature. Temperature gradient gel electrophoresis (TGGE) analysis was then performed in a buffer containing 50 mM TRIS pH=8.0, 100 mM NaCl, and 0.2 mM MgCl₂ as previously reported. A gradient temperature (40-80° C.) was applied perpendicular to the electrical current, and the experiment was run for 1 hr at 20 W. The gel was stained and visualized as described above. Quantified values of bands for each nanoparticle were divided by the sum of the total values in corresponding lanes, calculated by ImageJ. Melting curves were plotted with quantified data points using GraphPad Prism 8. Tₘ values were defined as the temperature at which 50% of the loaded nanoparticles dissociated.

Cell Culture

KB and HepG2 cells were obtained from American Type Culture Collection (ATCC). KB cells were grown and cultured in folate-free 1640 RPMI medium (ThermoFisher Scientific) containing both 10% (v/v) FBS and 100 U/ml penicillin at 37° C. in humidified air environment containing 5% CO₂.

Confocal Microscopy Imaging

KB cells were seeded on glass cover slips and cultured at 37° C. overnight. Alexa Fluor 647 labeled RNA nanoparticles were incubated with cells at a final concentration of 100 nM for 1 hr at 37° C. The slips were washed twice with PBS buffer followed by fixation using 4% formaldehyde. A 0.1% Triton X-100 (Sigma-Aldrich) in PBS buffer was used to treat the slips for 5 min, followed by subsequent cytoskeleton staining using Alexa Fluor 488 phalloidin (ThermoFisher Scientific) for 30 min at room temperature. After rinsing with PBS buffer, the cells were mounted with ProLong@ Gold Antifade Reagent (Life Technologies Corp.) containing DAPI for cell nucleus staining and imaged on Olympus FV3000 confocal microscope (Olympus Corp.). Data were acquired using Fluoview FV31S-SW.

In Vitro Apoptosis Assay

Cell apoptosis effect induced by RNA nanoparticles was evaluated using FITC Annexin V Apoptosis Detection Kit (BD Pharmingen) following manufacturer instructions. Briefly, 5×10⁴ KB cells were seeded on a 24-well plate overnight. RNA nanoparticles and free MTX were added into 24-well plate at a final concentration of 500 nM. After a 24 hr incubation at 37° C. in a humidified 5% CO₂ environment, cells were trypsinized to single cell suspension, washed with PBS buffer twice, and resuspended in 100 μL 1× Annexin V-FITC binding buffer. Following this 5 μL of Annexin V-FITC and 5 μL of PI were added into each sample and incubated at room temperature for 15 min. The samples were then transferred into flow tubes that contained 200 μL of 1× binding buffer for FACS analysis by FACS Calibur™ flow cytometer within 1 hr. Data were analyzed by FlowJo.

Flow Cytometry Analysis of the Binding of Motion Element-M-MTX to Cells

100 nM RNA nanoparticles with varying number of MTX harboring AFDye 647 were each incubated with 2×10⁵ KB cells at 37° C. for 1 hr respectively. After washing with PBS twice, the cells were resuspended in PBS. Flow cytometry analysis was performed by The Ohio State University Analytical Cytometry Shared Resource (ACSR) core facility to compare the binding efficacy of the fluorophore labeled RNA nanoparticles to different cells. The data was analyzed by FlowJo 7.6.1 software.

In Vivo Biodistribution of RNA NP

80 μL of 15 μM motion element, motion element-3MTX, motion element-18MTX, and motion element-28MTX sample fluorescently labeled with AFDye 647 were injected into 5-6 weeks old male BALB/c mice (Taconic) intravenously (IV) through the tail vein. Mice administered with PBS served as blank controls. Eight hours post-injection, mice were sacrificed, and their hearts, kidneys, livers, spleen, and lungs were collected and imaged for Cy5 fluorescent signal using an In Vivo Imaging System (IVIS) imager (Caliper Life Sciences). All animal experiments were housed and performed in accordance with the Subcommittee on Research Animal Care of the Ohio State University guidelines approved by the Institutional Review Board.

Urinary Sample Characterization of Motion Element-M-MTX

After injection of RNA samples into mice, hydrophobic sand was used to collect the mice urine within 1 hr of injection. 5 μL of urine from each group was loaded on 12% native PAGE for characterization, followed by imaging using Typhoon imaging system under Ethidium Bromide and Cy5 channel.

In Vitro Evaluation of Cytokine Induction by RNA NP

For the in vitro pro-inflammatory cytokine induction study, 2.5×10⁵ RAW 264.7 cells/well were seeded in a 24-well plate and cultured overnight. On the next day, 1 μM RNA NP and LPS were diluted in DMEM and added to cells for incubation in triplet. After a 16 hr incubation, cell culture supernatants were collected. TNF-a and IL-6 in the collected supernatants were examined by using Mouse ELISA MAX Deluxe sets (BioLegend, San Diego, CA, USA), following the manufacturer's instructions.

### Example 2. RNA Nanoparticles Solubilize and Targeted Deliver Hydrophobic SN38 for Colorectal Cancer Treatment

Results

Improvement of Water-Solubility of SN38 by Covalent Conjugation to RNA Strand

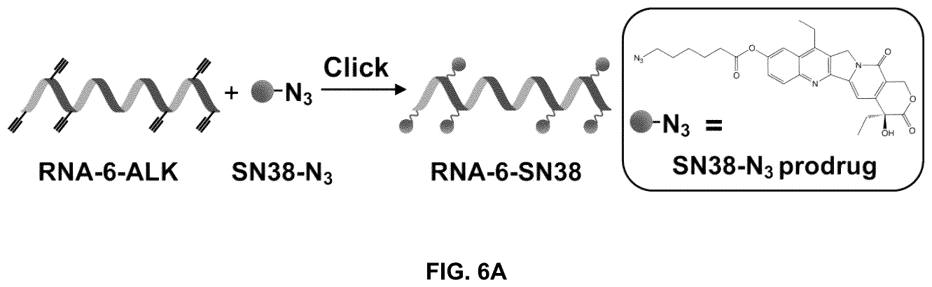

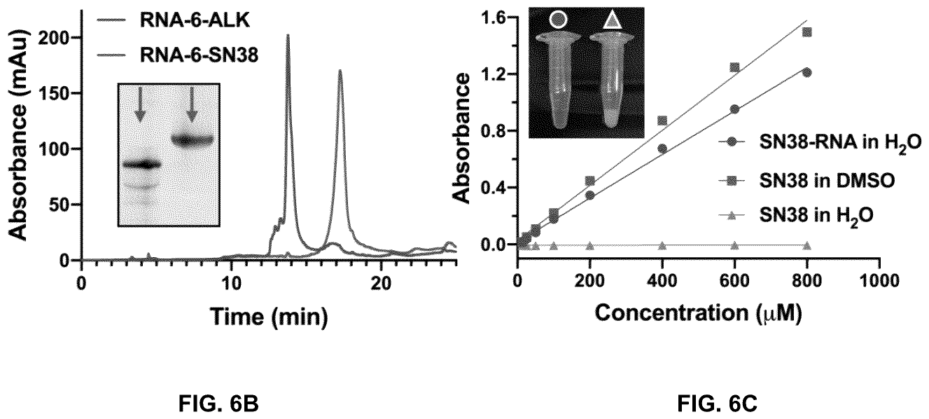

SN38 is a highly hydrophobic small molecule anticancer drug with poor solubility in water and in clinical formulations. To solve this problem, we conjugated SN38 to hydrophilic RNA strands via click reaction. SN38-azide (SN38-N₃) was synthesized with SN38 and AHA through esterification. 2′-Fluoro-modified RNA strand incorporated with six alkyne groups (RNA-6-ALK) was produced using 2′-O-propargyl and 5′-Hexynyl phosphoramidite by solid-phase synthesis. RNA-6-SN38 strand was prepared by the click reaction between SN38-N₃ with RNA-6-ALK (FIG. 6A). RNA-6-SN38 strands were then purified by RP-HPLC, and their quality were checked by gel electrophoresis and analytical HPLC (FIG. 6B). Dissolving RNA-6-SN38 conjugate in DEPC water yield clear solutions at increasing concentration from 12.5 μM to 800 μM (4.9-313.9 μg/mL) (FIG. 6C). The absorbance of SN38 in water remains at low level, which suggests the poor water solubility of SN38. In contrast, the absorbance of RNA-SN38 in water showed linear increase with concentration which indicates that RNA-SN38 has good solubility in water similar to the solubility of SN38 in DMSO (FIG. 6C).

Construction and Characterization of Thermostable Motion Element RNA Nanoparticles with SN38 and EpCAM Aptamer

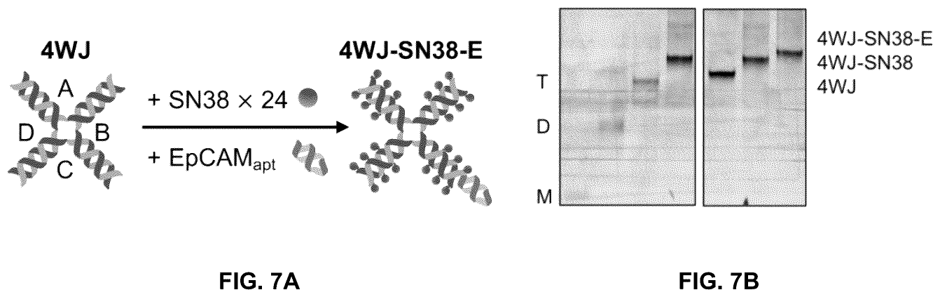

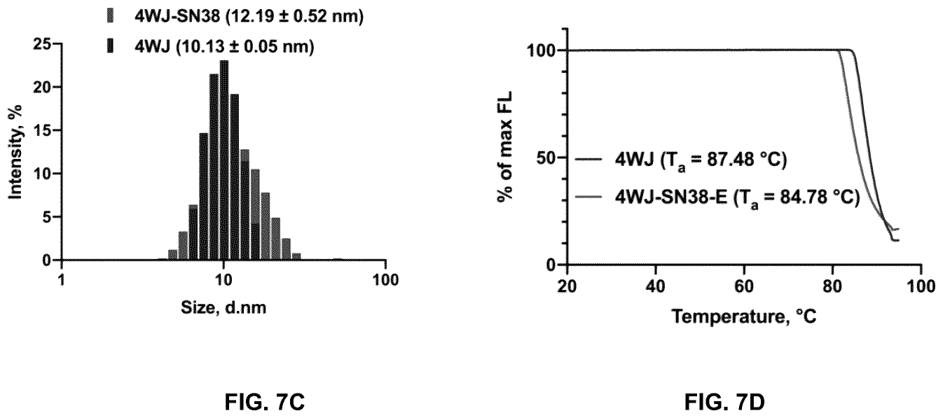

Thermostable motion element RNA nanoparticle was designed as the drug delivery platform with four 42-nt RNA strands, A, B, C, and D with 2′-Fluoro-modification and high GC content. To construct motion element-SN38-EpCAMapt (motion element-SN38-E) RNA nanoparticles, four RNA strands were each conjugated with 6 copies of SN38, and B strand was extended with an EpCAM aptamer (EpCAMapt) for targeting (FIG. 7A). motion element and motion element-SN38-E RNA nanoparticles were self-assembled by mixing and annealing four composing RNA strands at equal molar ratio. Both motion element and motion element-SN38-E RNA nanoparticles were assembled with high efficiency indicated by gel electrophoresis (FIG. 7B). With the attachment of SN38 and EpCAMapt, the size of motion element increased from 10.13 nm to 12.19 nm while the zeta potential remains negative, which was measured by DLS (FIG. 7C). Due to the incorporation of 2′F-C and U as well as the high GC content, both motion element and motion element-SN38-E exhibited high thermostability, with the annealing temperature above 80° C., which is much higher than physiological condition (37° C.) (FIG. 7D).

Thermostability of functionalized motion element RNA nanoparticles including motion element-SN38 and motion element-SN38-E was studied using motion element for comparison. The melting profile showed that the incorporation of 24 copies of SN38 to motion element slightly decreased it thermostability, but the Tm is still higher than 70 C, which allows it to stay stable in physiological conditions. The annealing profile further confirmed this result that SN38 decrease the thermostability of motion element, but the Ta is still higher than 80 C.

In Vitro Cell Binding and Internalization of RNA Nanoparticles Mediated by the EpCAM Aptamer

Specificity is one of the key considerations in the drug delivery field. Targeted delivery can not only improve therapy outcome but also reduce normal organ and tissue accumulation, thus lowering side effects. In this study, we incorporated EpCAM RNA aptamer, which can form a defined 3D structure to bind to the EpCAM highly expressed on the surface of HT29 cell. To evaluate the specific targeting efficiency, RNA nanoparticles labeled with AFDye 647 were incubated HT29 cells for flow cytometry analysis. motion element-SN38-EpCAM showed strong binding to HT29 cells, while motion element itself showed no binding to cells due to its negative charge.

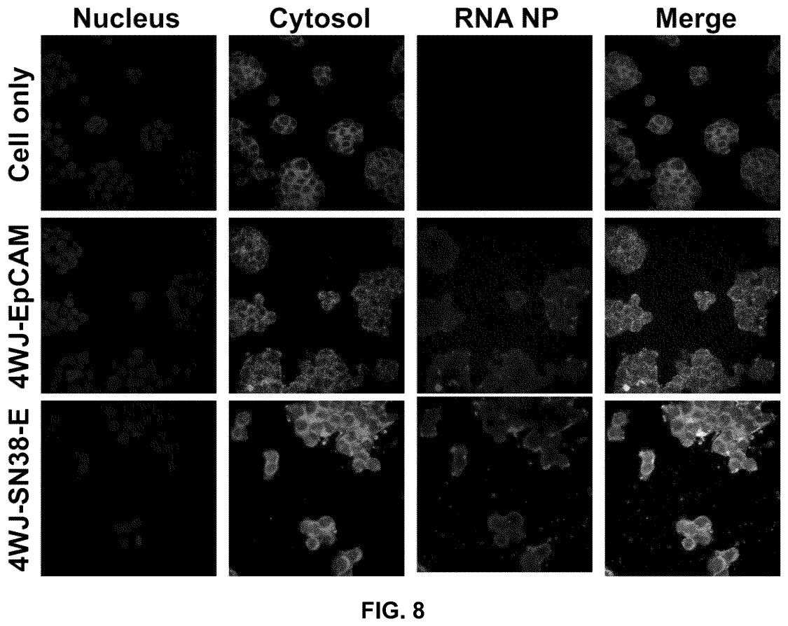

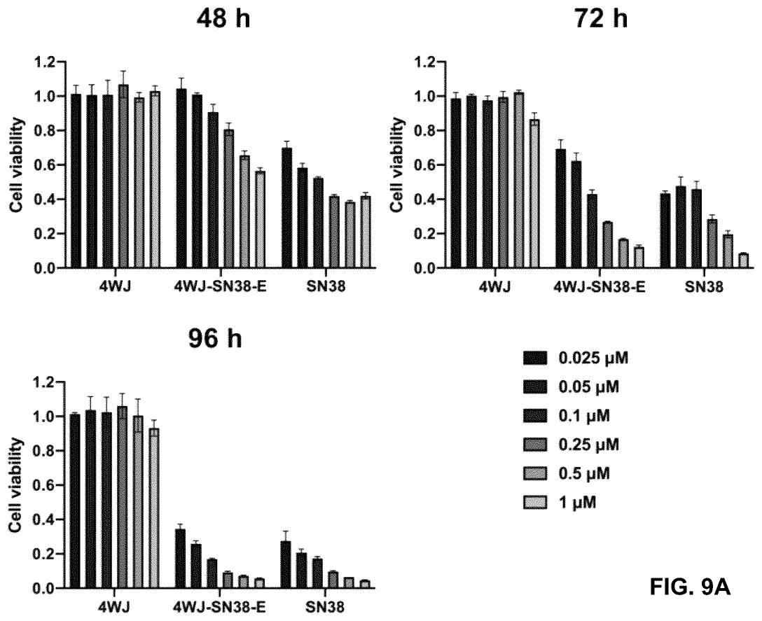

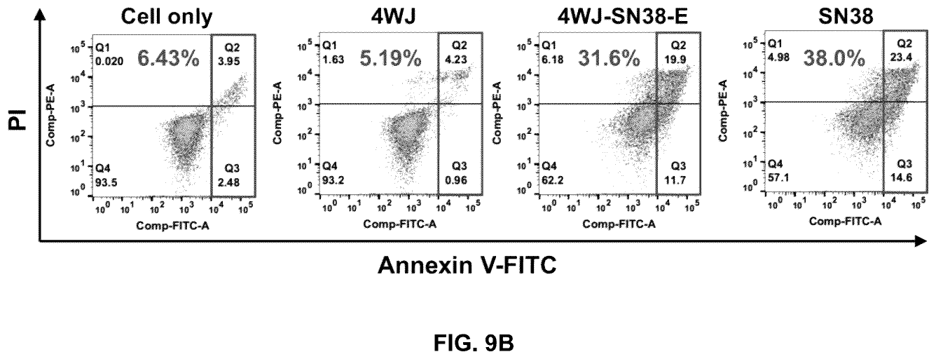

In addition, confocal microscope images were taken to study whether EpCAM aptamer decorated RNA nanoparticles can enter HT29 cells (FIG. 8). The images demonstrated that the fluorescence of motion element-SN38-EpCAM nanoparticles is strongly overlapped with the cell cytosol. motion element-SN38-E treated cells showed high intracellular fluorescence signal, while signals for motion element is low. These results together demonstrated the EpCAMapt displaying RNA nanoparticles specifically bind to EpCAM-overexpressed tumor cells and are further internalized into the cells efficiently by receptor-mediated endocytosis. The enhanced internalization profile provides a foundation for motion element-SN38-EpCAM to be applied in CRC targeting and therapy.

In Vitro Cytotoxicity and Immunogenicity

To determine the cytotoxicity of RNA-SN38 nanoparticles, dose-dependent MTT assay was performed. The cell viability results demonstrated RNA-SN38 nanoparticles inhibited HT29 cell growth in a concentration dependent manner, while RNA nanoparticles without SN38 did not show obvious inhibition. These results demonstrated that SN38 was released from the RNA nanoparticles and retained its pharmacological activity to inhibit tumor cell growth. Consistent with the MTT assay, PI and FITC Annexin V double staining analysis confirmed tumor cell apoptosis induced by RNA-SN38 nanoparticles. The results showed % and % of cells underwent apoptosis after being treated with motion element-SN38-E and SN38.

Immunogenicity is one of the concerns in the use of nanoparticles fabricated with biomaterials. To evaluate immunogenicity of RNA nanoparticles, production of pro-inflammatory cytokines, including TNF-α (tumor necrosis factor α) and IL-6 (interleukin-6) was investigated in vitro. Tumor necrosis factorα (TNF-α) is cytokine involved in systemic inflammation and one of the cytokines that make up the acute phase reaction. Interleukin 6 (IL6) is an interleukin that acts as both a pro-inflammatory cytokine and an anti-inflammatory myokine. IL6 is secreted to stimulate immune response during infection. The results showed that motion element-SN38-EpCAM at the therapeutic concentration (100 nM) induced negligible TNF-α nor IL-6 production compared to LPS (lipopolysaccharide) positive control while incubating with mouse macrophage like RAW 264.7 cells in vitro. The low induction of cytokine suggests that RNA as biomaterial is biocompatible and relative safe.

In Vivo Tumor Inhibition Study

Given the demonstration of specific tumor cell binding and apoptosis effect, we studied the tumor suppression in tumor xenograft model. The therapeutic effect of motion element-SN38-EpCAM nanoparticles was validated by a tumor inhibition study in an orthotropic CRC model in nude mice. After the tumor grow to approximately 50 mm, mice bearing HT29 xenograft were randomly divided into four groups of PBS, SN38, motion element-SN38, and motion element-SN38-E. Samples were administrated via IV injection at the dose of 2 mg/kg (SN38/body weight) on day 0, 3, 6, 9, and 12 for a total of five injections. motion element-SN38-EpCAM and SN38 showed similar tumor suppression. Both SN38-RNA nanoparticles did not cause any fatality or obvious weight changes during experiments, suggestive of a favorable safety profile of the SN38-RNA nanoparticles. Due to the recognition between EpCAM aptamer and its overexpressed receptor on HT29 cells. motion element-SN38-EpCAM outperformed motion element-SN38 in tumor suppression by about % more reduction in tumor weight.

### DISCUSSION

In summary, RNA nanoparticles were used for solubilizing hydrophobic antitumor drug and for targeted delivery in a colorectal cancer xenograft model. High loading of poor soluble SN38 to the hydrophilic RNA nanoparticles dramatically improved drug solubility in physiological conditions while maintaining therapeutic effect. The resulting therapeutic RNA nanoparticles had the advantage of well-defined structure, precise drug loading, high thermal and chemical stability, and targeted delivery. The potent SN38 was enzymatically released from the RNA vehicles by esterase in vivo, triggering tumor cell apoptosis and inhibiting tumor growth effectively. The in vitro and in vivo data collectively demonstrated the feasibility of RNA nanoparticles for the safe and efficacious targeted delivery of hydrophobic antitumor drugs.

Materials and Methods

Synthesis of SN38-N3 Prodrug

To synthesize SN38-Azide, 1:2:2:1 eq. of 7-Ethyl-10-hydroxycamptothecin (SN38) (100 mg), 1-Ethyl-3-(dimethylaminopropyl) carbodiimide hydrochloride (EDC·HCl), 6-Azido-hexanoic acid, and 4-(Dimethylamino) pyridine (DMAP) were weighted and added into a round bottom flask. The mixture was dissolved in 10 mL Dichloromethane (DCM) and reacted with stirring at room temperature for 24 h. The reaction solution was filtered and concentrated on a rotary evaporator. The concentrated reaction was subsequently purified by CombiFlash column chromatography under serial solvent wash with DCM as solvent A and Ethyl acetate as solvent B. Fractions were collected and analyzed by thin-layer chromatography (TLC) and the ones containing pure product were combined and dried. The purified final product SN38-Azide was characterized by Nuclear Magnetic Resonance (NMR) Spectroscopy.

Synthesis of RNA-SN38 Strand Via “Click Chemistry”

All RNA strands were synthesized by ASM-800ET DNA/RNA synthesizer (Biosset) using A, G 2′-Fluoro C and 2′-Fluoro U phosphoramidites (ChemGenes). 5′-Hexynyl and 2′-O-propargyl phosphoramidites (Glen Research, ChemGenes) were incorporated into RNA-ALK strands for drug conjugation. The sequences of all RNA strands are listed in the SI table (Table S1).

Conjugation of SN38-N3 to RNA-6-alkyne (RNA-6-ALK) strand was performed using copper(I)-catalyzed alkyne-azide cycloaddition (“Click chemistry”). RNA-6-ALK strand dissolved in diethyl pyrocarbonate-treated water (DEPC-H2O) were thoroughly mixed with SN38-N3 dissolved in 3:1 (v/v) dimethyl sulfoxide/tert-butanol (DMSO/t-BuOH). Freshly prepared “click solution” (a mixture of CuBr/TBTA at a 1:2 molar ratio in DMSO/t-BuOH) was then added with the final molar ratio of RNA/SN38/Cu+ at 1:15:20. After incubation at room temperature overnight, the reaction product was characterized by 16% 8 M urea PAGE in TBE buffer (89 mM Tris base, 200 mM boric acid and 2 mM EDTA) at 200 V for 1 h. After staining by ethidium bromide (EtBr, Sigma-Aldrich) and washing, gel was visualized and analyzed by a Li-Cor Odyssey Fc imaging system.

RNA-SN38 was first purified by ethanol precipitation with 1/10 volumes of 0.3 M sodium acetate and 2.5 volumes of 100% ethanol followed by resuspension in DEPC-H2O. It was subsequently purified by Ion-Pair Reverse Phase high performance liquid chromatography (HPLC) using PLRP-S 4.6×250 mm 300 A column (Agilent Technologies). Fully conjugated RNA-SN38 was separated from unreacted and not fully reacted RNA-ALK strands under gradient elution with 0.1% Triethylammonium acetate (TEAA) in H2O as solvent A and 0.1% TEAA in 75% Acetonitrile (AcN) as solvent B. Fractions were collected and analyzed by PAGE and the ones containing pure product were combined, dried and resuspended in DEPC-H2O.

Evaluation of Improved Water Solubility of SN38 after Conjugation to RNA

Three solutions were prepared, RNA-SN38 in DEPC H₂O, SN38 in DEPC H₂O, and SN38 in DMSO. The absorbance at 366 nm of these three solutions with different concentrations (12.5-800 μM) were measured by NanoDrop 2000 Spectrophotometer (ThermoFisher). The absorbance for all three solutions were plotted against SN38 concentration, respectively. The picture of RNA-SN38 and SN38 in DEPC H2O at 800 μM were taken for comparison of clarity and turbidity.

Assembly of 2′F Modified Motion Element RNA Nanoparticles

To assemble motion element RNA nanoparticle, four single RNA strands (A, B, C, and D) were mixed at equal molar ratio in TES buffer (50 mM Tris pH=8.0, 50 mM NaCl, 1 mM EDTA). The mixture solution was subjected to a one-hour annealing program that starts with denaturing at 95° C. for 5 min followed by cooling to 4° C. in Mastercycler (Eppendorf). motion element-SN38 RNA nanoparticle was generated using the same procedure with single RNA-SN38 strands. After annealing, RNA nanoparticles were checked by 10% native PAGE in TBE buffer at 120 V for 1 h to check the assembly efficiency. After staining by ethidium bromide (EtBr, Sigma-Aldrich) and washing, gel was visualized and analyzed by a Li-Cor Odyssey Fc imaging system.

Dynamic Light Scattering (DLS)

The hydrodynamic diameter and zeta potential of the RNA nanoparticles with the concentration of 10 μM in TES buffer were measured using Zetasizer Nano-ZS (Malvern Instrument) at room temperature. Size distributions were plotted with data points using GraphPad Prism.

Measurement of Melting Profile by Thermal Gradient Gel Electrophoresis (TGGE)

The melting profile of RNA nanoparticles in TES buffer was measured by TGGE in TBE buffer. RNA nanoparticles with a final concentration of 1 μM were subjected to 10% native PAGE gel and run vertically at room temperature for 10 min followed by run horizontally on TGGE equipment for 60 min with the temperature gradient (Left to right: 39.6-79.6° C.). After staining by EtBr and washing, gels were imaged by a Li-Cor Odyssey Fc imaging system and quantified by ImageJ.

Measurement of Annealing Profile

The annealing profile of RNA nanoparticles in TES buffer was measured by LightCycler® 480 (Roche). RNA nanoparticles with a final concentration of 2.5 μM were added with 10×SYBR Green II (Invitrogen) into 96-well plate. The annealing process starts denaturing at 95° C. for 5 min followed by a gradual cooling process at a rate of 0.11° C. s-1 to 20° C. The percentage of maximum fluorescent signal was plotted against temperature to indicate the annealing profile.

Cell Culture

HT29 cells were cultured in MyCoy's 5A medium (Thermo Fisher Scientific) supplemented with 10% fetal bovine serum (FBS) at 37° C. in humidified air containing 5% CO₂. Mouse macrophage-like RAW 264.7 cells were cultured in DMEM (Thermo Fisher Scientific) supplemented with 10% fetal bovine serum (FBS) at 37° C. in humidified air containing 5% CO2.

In Vitro Cell Binding Assay Using Confocal Microscopy

HT29 cells were grown on glass slides in 24-well plate and cultured at 37° C. overnight. RNA nanoparticles labeled with AFDye 647 were incubated with 2×10⁵ HT29 cells with a final concentration of 200 nM at 37° C. for 2 h. The cells were washed twice with PBS and fixed by 4% paraformaldehyde (PFA). After washing with PBS, the fixed cells were stained by Alexa Fluor 488 Phalloidin (Life Technologies) for cytoskeleton and Fluoroshield Mounting Medium with DAPI (Abcam) for nucleus, respectively. The cells were then imaged and analyzed by FluoView FV3000-Filter Confocal Microscope System (Olympus Corp).

In Vitro Cytotoxicity Using MTT Assay

HT29 cells were plated into 96-well plate and cultured at 37° C. overnight. The cytotoxicity of RNA nanoparticles and SN38 was studied by CellTiter 96® Non-Radioactive Cell Proliferation Assay (Promega). RNA nanoparticles and free SN38 were each incubated with HT29 cells in 100 μl cell medium containing 10% FBS at 37° C. for 48 hours. 15 μl of MTT dye solution was added to each well containing 100 μl of cells in cell medium and incubated for 3 hours at 37° C. in a humidified, 5% CO2 atmosphere. The cell medium supernatant was discarded after dye incubation and 100 μl DMSO was then added to solubilize the formazan product. The absorbance at 570 nm was recorded using a 96-well plate reader (BioTek).

In Vitro Apoptosis Assay

HT29 cells were plated into 24-well plate and cultured at 37° C. overnight. The cell apoptosis induced by RNA nanoparticles and free SN38 was studied by FITC Annexin V Apoptosis Detection Kit (BD Pharmingen). RNA nanoparticles and free SN38 were each incubated with HT29 cells in 400 μl cell medium containing 10% FBS at 37° C. for 24 hours. HT29 cells were then trypsinized to single cell suspension and washed with PBS. The cells were re-suspended in 100 μl 1× Annexin V-FITC binding buffer. Then 2 μl Annexin V-FITC and 2 μl propidium iodide (PI) were added into each sample and incubated at room temperature for 15 min. The samples were transferred to flow tubes which contained 150 μl 1× binding buffer for fluorescence-activated cell sorting (FACS) analysis by LSR II Flow Cytometer (Becton Dickinson), and the data were analyzed by FlowJo 7.6.1 software.

In Vitro Cytokines Induction Assay

RAW 264.7 cells were plated into 24-well plates and cultured at 37° C. overnight. RNA nanoparticles and lipopolysaccharide (LPS, 5.5 μg/mL, equal amount as 100 nM motion element RNA nanoparticles) were each incubated with macrophage cells in 200 μl Opti-MEM cell medium (Thermo Fisher Scientific) at 37° C. for 16 hours. The supernatants of cell medium were collected and frozen at −80° C. for further analysis. The TNF-α and IL-6 in diluted supernatants were measured by Mouse ELISA MAX Deluxe sets (BioLegend) following manufacturer's protocols.

Tumor Xenograft Animal Model

All animal procedures were performed in accordance with Subcommittee on Research Animal Care of The Ohio State University guidelines approved by the Institutional Review Board. The protocol for this animal experiment was approved by the Institutional Animal Care and Use Committee (IACUC) of The Ohio State University. HT29 cell derived tumor xenograft mice model were established by subcutaneously injecting 2×10⁶ HT29 cells in 100 μl PBS into each female athymic nu/nu mice (4-6 weeks old).

In Vivo Tumor Inhibition

Mice with established tumor nodules were randomly divided into four groups (n=5 biologically independent animals). Samples were administrated by i.v. injection in a total of 5 doses (2 mg kg-1, SN38 per body weight) every 3 days for 15 days. Tumor volume was monitored everyday by caliper, calculated as (length×width2)/2, and mouse weight were monitored every day. Curves were plotted with data points using GraphPad Prism. On day 15, the mice were sacrificed followed by tumor extraction. Tumors were weighted, and result was plotted with data points using GraphPad Prism. Data were statistically analyzed by two-tailed unpaired t-test and presented as mean±SD; *p<0.05; **p<0.01; ****p<0.0001

Statistics

Each experiment was repeated independently for at least three times. The results are presented as mean±standard deviation (S.D.). Statistical differences were evaluated using two-tailed unpaired t-test using GraphPad software. p<0.05 was considered statistically significant. p<0.001 is denoted as *** and p<0.0001 is denoted as ****.

### Example 3. RNA Nanoparticles to Carry Cancer Specific Targeting Ligands and Radioisotopes for Cancer PET Imaging

Results

DCL-Azido Preparation and Purification

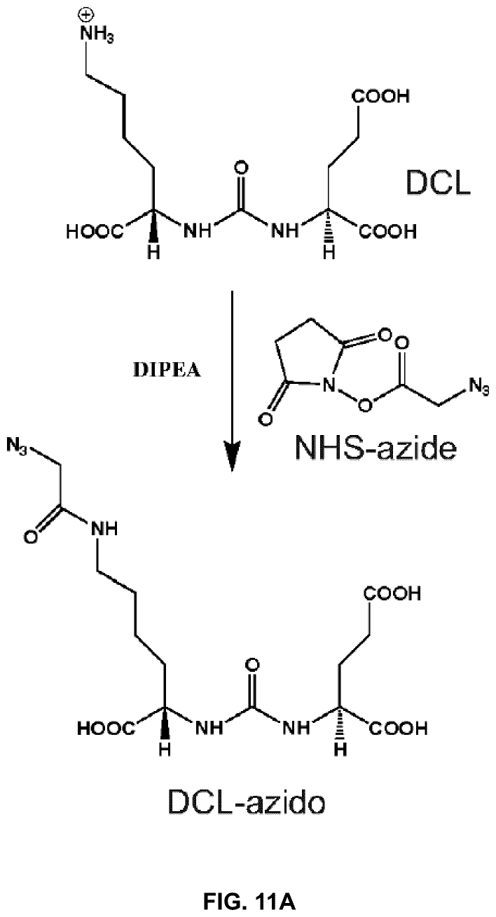

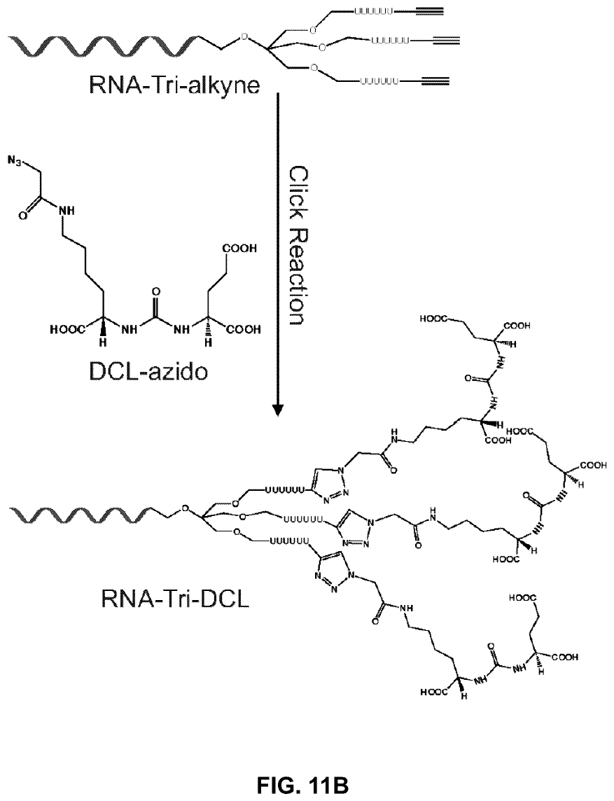

As PSMA is specifically overexpressed in prostate cancer and metastases cells, it has been used as an ideal target for prostate cancer. PSMA, also known as folate hydrolase I, is a transmembrane with 750 amino acid and type II glycoprotein (Afshar-Oromiehet, A, al. J Nucl Med. 2016 57:79S-89S). Structural studies revealed that the binding pocket of PSMA contains two zinc ions located on the amino acid catalytic site. Located in proximity of this amino acid site, a funnel-shaped tunnel with a depth of approximately 20 Å and a width of about 9 Å was observed (Banerjee, S. R. et al. Oncotarget. 2011 2:1244-1253; Sanna, V. et al. J Med Chem. 2011 54:1321-1332; Siva, S. et al. Nat Rev Urol. 2020 17:107-118). In this study, we selected a urea-based motif containing lysine (DCL, FIG. 11) as a PSMA-targeting ligand. The DCL contained a primary amine that allowed for modification of bio-orthogonal reactive group. To enable the conjugation to RNA, the DCL was modified with azido group via N-HydroxySuccinimide (NHS) ester reaction as shown in FIG. 11A. A bi-functional linker NHS-azido was used to mediate the conjugation of DCL on alkyne-modified RNA.

Implementation of Multivalent DCL on RNA Nanoparticles

To conjugate the DCL on RNA nanoparticles, RNA was modified with a varying number of alkyne groups at the 5′-end utilizing the doubler and trebler branching phosphoramidite. By creating a branching point, six uracil nucleotides were incorporated as a long linker to impart the flexibility of conjugated ligands (FIG. 11B). All other pyrimidines (C&U) were modified with 2′-fluorine (2′-F) to confer enzymatic stability on RNA. Then the DCL-conjugated strand was used as a composite strand for nanoparticle assembly. In this study, we utilized a new thermodynamically stable RNA scaffold re-engineered from naturally occurring SF5 packaging RNA motif composed of three oligomers (i.e. SF5 a, b and c) (Hao, Y. et al. RNA. 2014 20:1759-1774). To increase the thermodynamic stability of nanostructure, the RNA cores were extended with 11 base pairing (bp) on each helix (H1, H2, and H3). Assembly of RNA complex was easily achieved by mixing equimolar ratio of motion element, a, b, c strands followed by an annealing process.

PSMA RNA aptamer was used as a control to evaluate the targeting capability of DCL conjugated RNA nanoparticles that has been reported for prostate cancer targeting previously (Binzel, D. W. et al. Mol Ther. 2016 24:1267-1277). The motion element-a of SF5 was designed to carry targeting ligand (DCL or PSMA aptamer) while motion element-b and motion element-c were conjugated with fluorescent probe or radioisotope chelator. Therefore, RNA nanoparticles harboring different ligands by changing the composite strands with varying ligands were obtained.

Characterization of RNA Nanoparticles with PSMA-Targeting Ligands

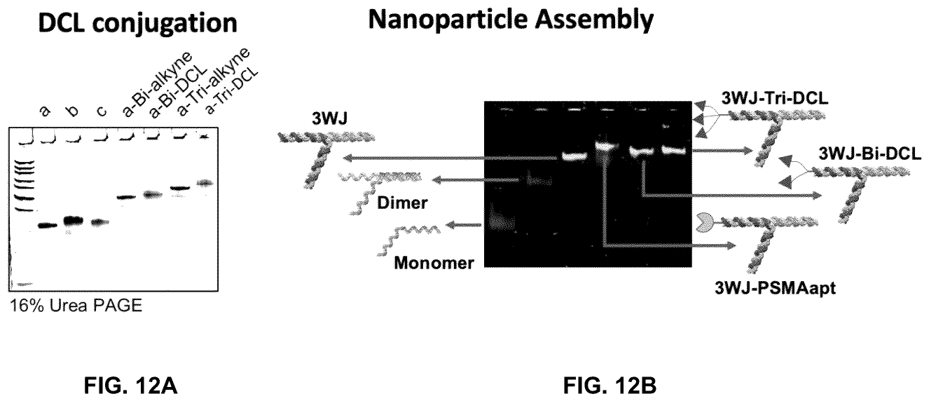

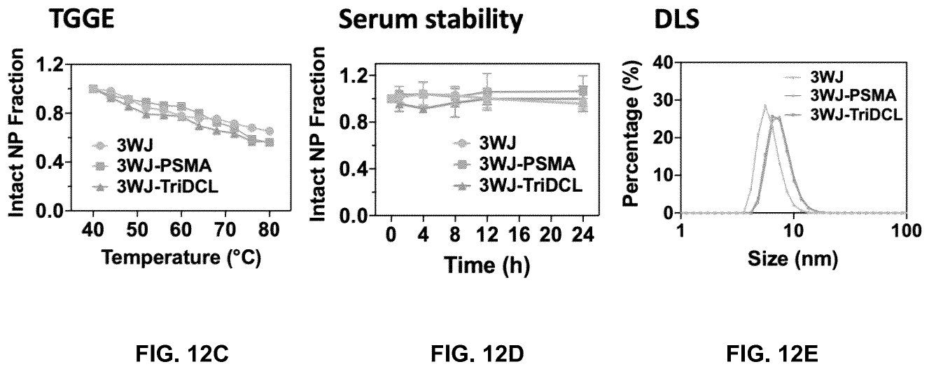

As three individual RNA oligos were incorporated into one nanoparticle complex, a stepwise assembly characterization by 16% native PAGE was performed (FIG. 12A). As shown in FIG. 12B, gel mobility shift was observed from monomer, dimer to RNA trimer complex. Assemblies of RNA nanoparticles containing bivalent DCL (motion element-Bi-DCL), trivalent (motion element-Tri-DCL) and PSMA aptamer (motion element-PSMAapt) were observed with slower gel migration, indicating the successful formation of motion element-based complex. As the physicochemical properties of RNA nanoparticles playing critical roles in governing the in vivo behavior, the thermodynamic stability, enzymatic stability, and size of these RNA nanoparticles were further characterized as described in detail below.

The thermodynamic stability of the PSMA targeting RNA nanoparticles was assayed using temperature-gradient gel electrophoresis (TGGE) (Benkato, K. et al. Methods Mol Biol. 2017 1632:123-133). Temperature gradient was applied perpendicular to the electrical current to determine the melting temperature of the RNA complex (FIG. 12C). With an increasing temperature from 40 to 80° C., more than 50% of all RNA complex remained intact, indicating the high melting temperature (Tm). Similar trend was observed in motion element-Tri-DCL and motion element-PSMA nanoparticles, which suggested that conjugating DCL ligands or PSMA aptamer did not hinder the stability of the RNA core. This result demonstrated the high thermodynamic stability of motion element-based constructs, making it suitable for in vivo applications.

Resistance to enzymatic degradation is another key factor to consider when using RNA as delivery platform. To increase the enzymatic stability of RNA nanoparticles, 2′-fluorine pyrimidines (C & U) in the RNA synthesis was introduced. The chemical stability of RNA nanoparticles by incubating with 10% fetal bovine serum (FBS) was assessed. Samples then were run on polyacrylamide gels; and the band corresponding to the intact assembled RNA nanoparticles was quantified. Results showed that all RNA nanoparticles remained stable up to 24 h when incubated with 10% FBS at 37° C. (FIG. 12D).

Previous study suggested the importance of the size of nanoparticles in determining the in vivo behavior (Jasinski, D. L. et al. Mol Ther. 2018 26:784-792). The hydrodynamic size of RNA nanoparticles at around 10 nm have been demonstrated to be suitable for in vivo tumor targeting with longer circulation time and less liver accumulation. Herein, dynamic light scattering (DLS) assays were conducted on the constructed motion element, motion element-Tri-DCL and motion element-PSMA nanoparticles. As a result of ligand conjugation, the diameter of motion element-Tri-DCL and motion element-PSMA were increased to 9.5±1.2 nm and 9.8±1.4 nm, respectively, from 7.6±1.7 nm (the diameter of motion element) (FIG. 12E). This result showed that motion element-Tri-DCL and motion element-PSMA may have a better efficiency on cancer therapy because of their size.

In Vitro Cellular Targeting and Internalization

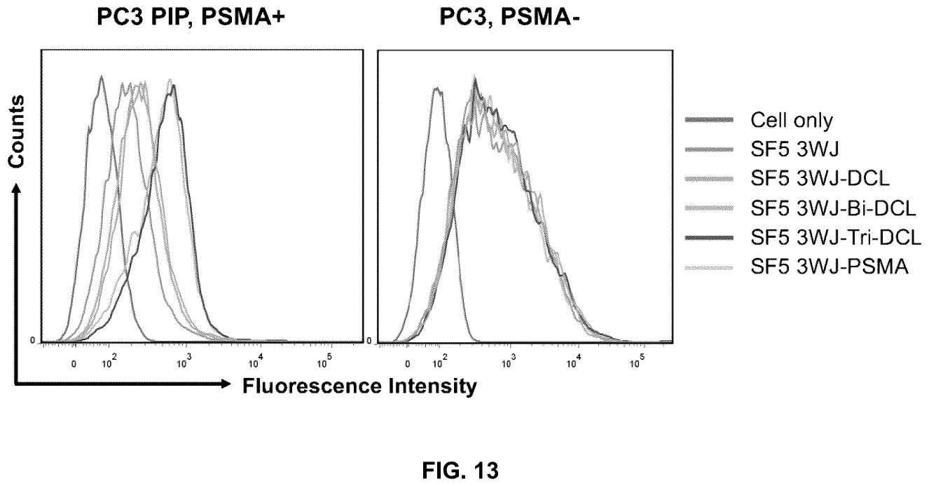

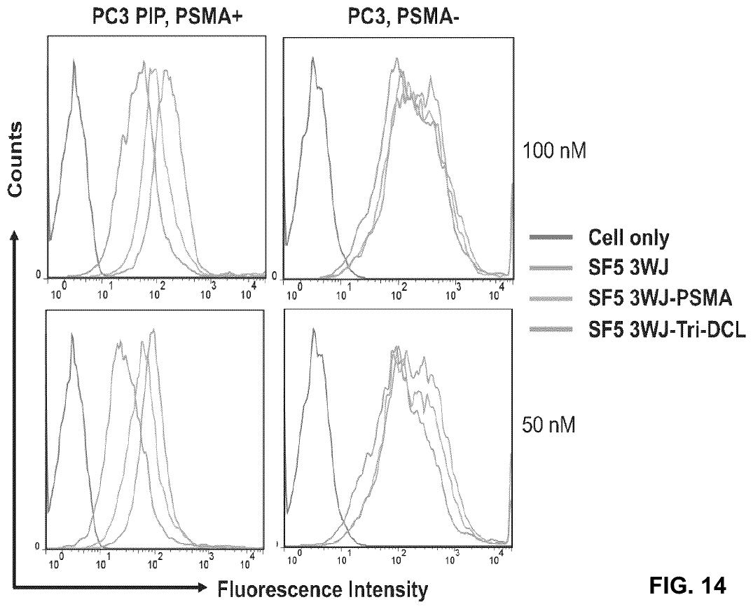

To evaluate the cellular targeting capability of RNA nanoparticles, flow cytometry analysis were performed. PSMA overexpressed PC3 PIP cell line was used to evaluate the specific binding of RNA nanoparticles to PSMA. Intriguingly, the binding affinity of motion element-DCL and motion element-PSMA showed binding enhancement compared to RNA nanoparticles, while both motion element-Bi-DCL and motion element-Tri-DCL had the strongest binding to PC3 PIP cells (FIG. 13, Left). In the negative control of PC3 cell line, no significant difference was observed among all groups, suggesting the PSMA-targeting binding by DCL- and PSMA-conjugated RNA nanoparticles (FIG. 13, Right). Particularly, the bivalent and trivalent DCL imparted the strongest enhancement in the specific PSMA-targeting binding. To further compare the trivalent DCL with PSMA aptamer, cellular binding assays at two different concentrations were conducted by using flow cytometry (FIG. 14). At both 50 nM and 100 nM concentration, the motion element-Tri-DCL showed strongest binding to PSMA overexpressed PC3 PIP cells, demonstrating the feasibility of using trivalent DCL for enhanced targeting to prostate cancer.

Internalization of RNA Nanoparticles to PSMA+ and PSMA− PC3 Cells

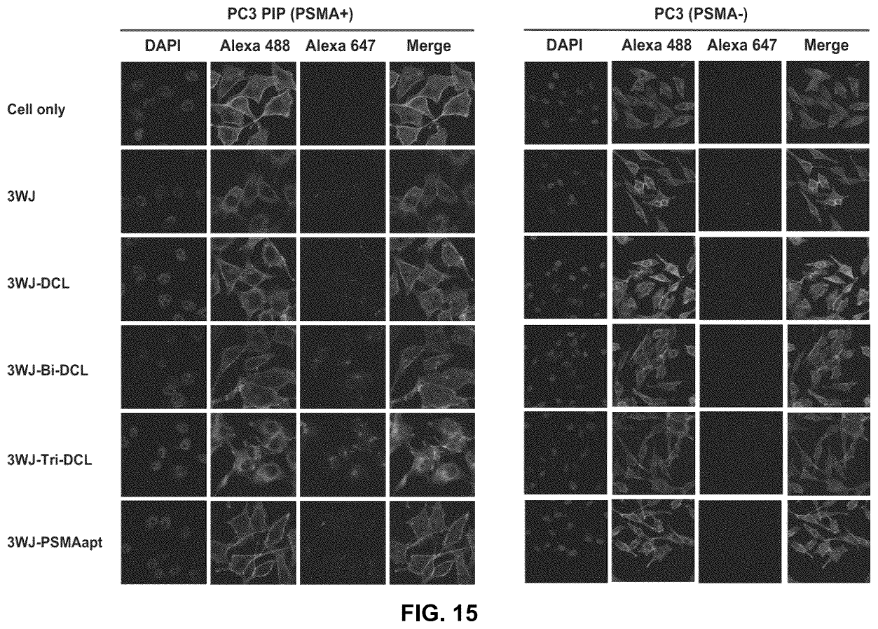

To evaluate whether the PSMA-targeting ligand would facilitate the internalization of RNA nanoparticles into cells, cell internalization assay was carried out by confocal microscopy imaging. As shown in FIG. 15, motion element-Bi-DCL and motion element-Tri-DCL exhibited substantial cell internalization. In contrast, motion element-DCL and motion element-PSMA nanoparticles showed less cellular uptake. Confocal microscopy images scanned under three separate emission channels clearly showed the difference in cellular uptake among all RNA nanoparticles groups. As the results suggested a strong correlation with DCL valency, it is arguably evidenced that an increased valency of DCL facilitated the receptor-mediated cellular uptake in vitro.

In Vivo Biodistribution Profile of RNA Nanoparticles

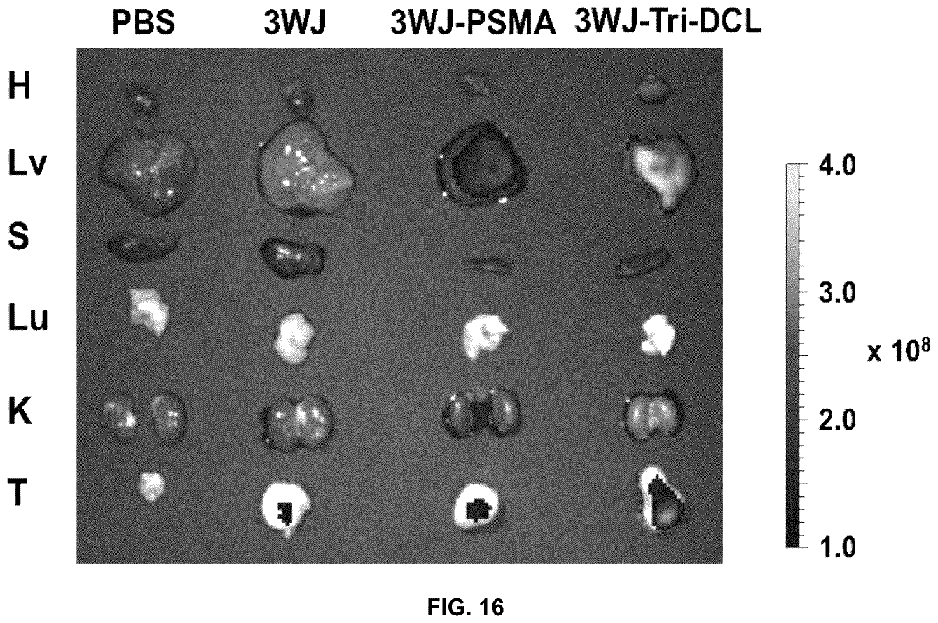

To compare the in vivo targeting effect and organ biodistribution, fluorescent ex vivo organ imaging was performed after systemic administration of fluorescently labeled RNA nanoparticles in mice through the tail vein. At 8 h post-injection, vital organs and tumor were taken out from mice for fluorescent imaging. In the PC3 PIP xenograft-bearing nude mice, motion element-Tri-DCL showed substantial tumor accumulation compared to RNA and motion element-PSMA nanoparticles. However, the liver accumulation was also found to be more significant in motion element-Tri-DCL group (FIG. 16). Interestingly to find that the motion element-PSMA also exhibited liver accumulation which might contributed by the increased overall hydrodynamic size.

Radiolabeling of RNA Nanoparticles with High Chelation Efficacy

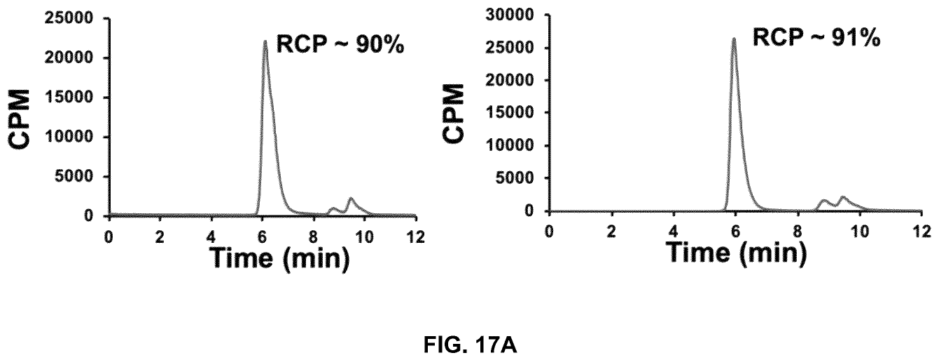

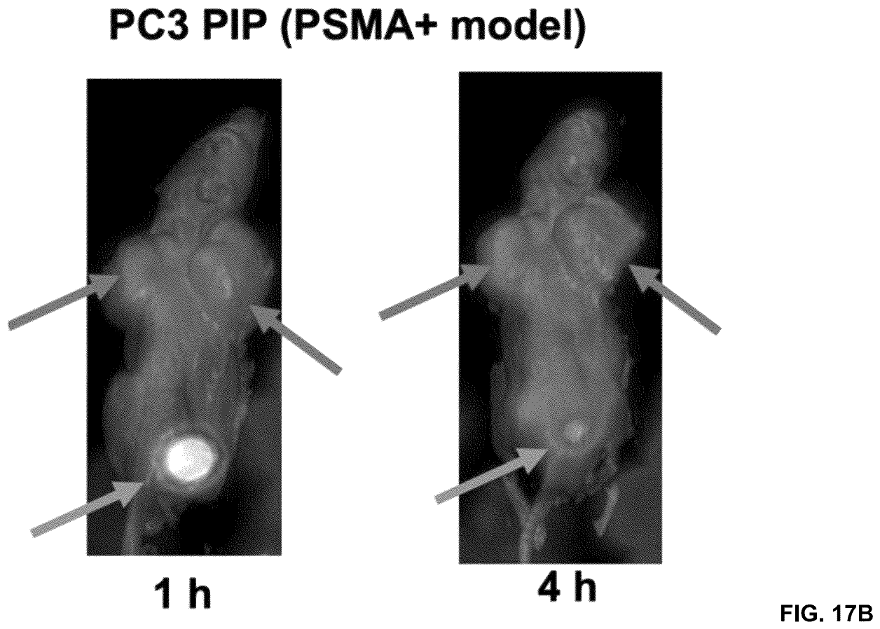

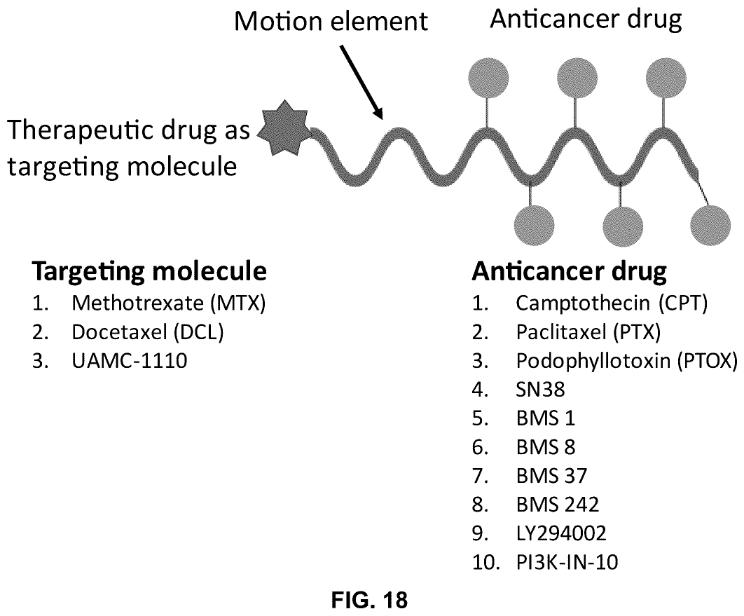

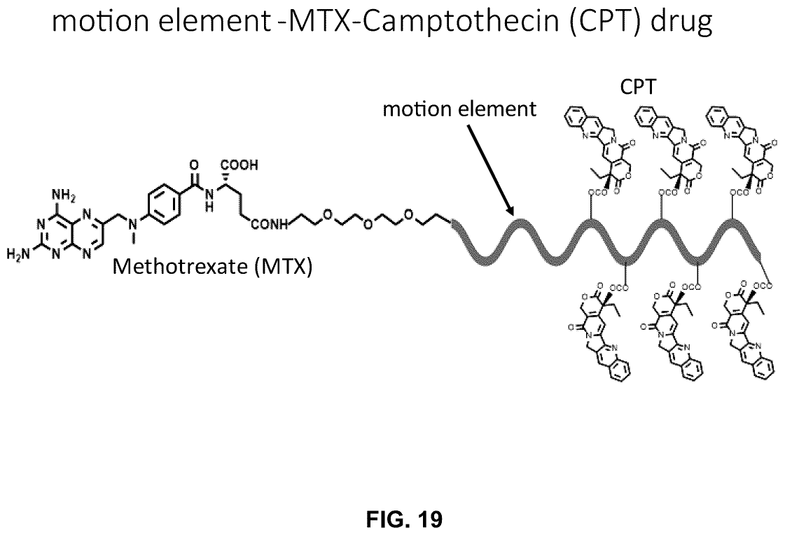

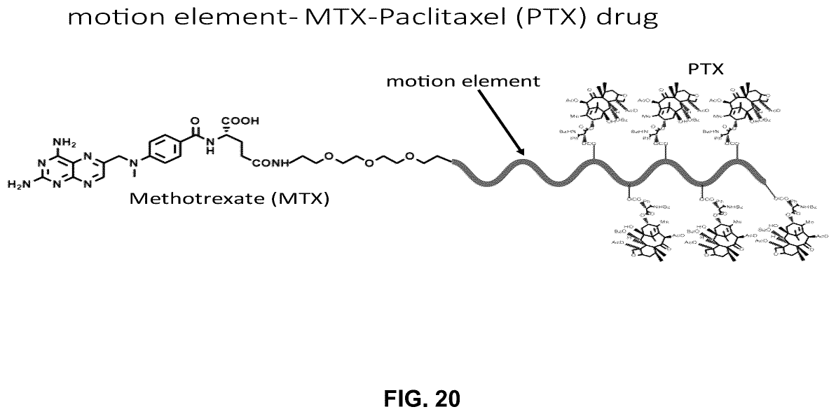

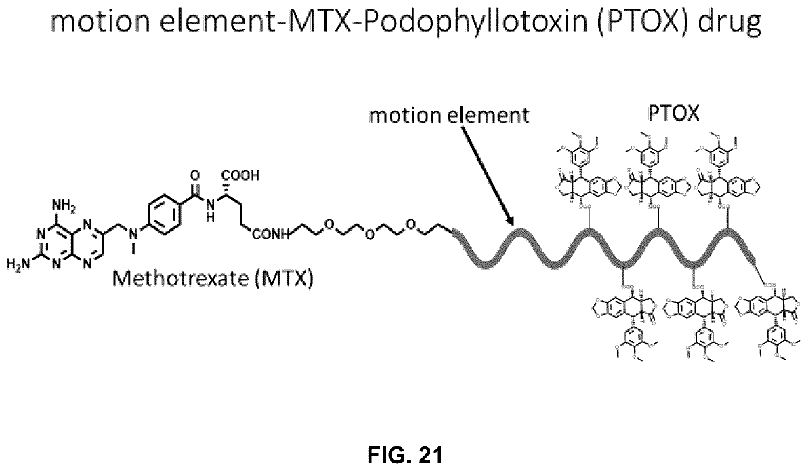

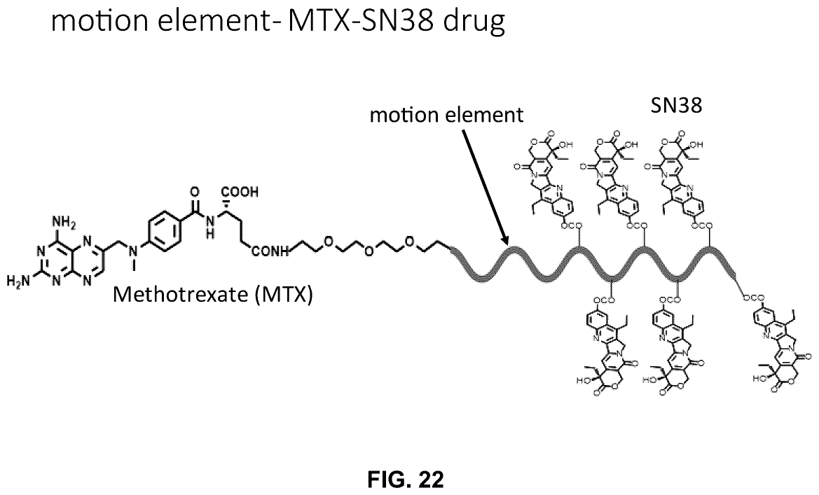

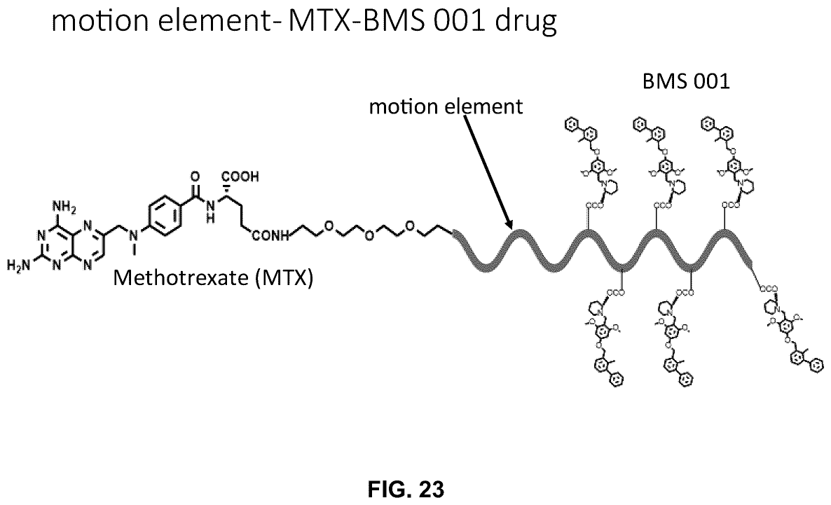

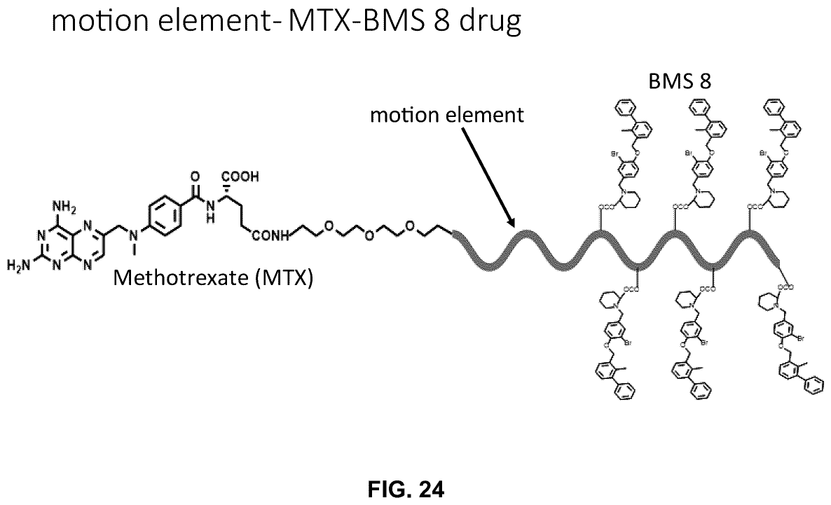

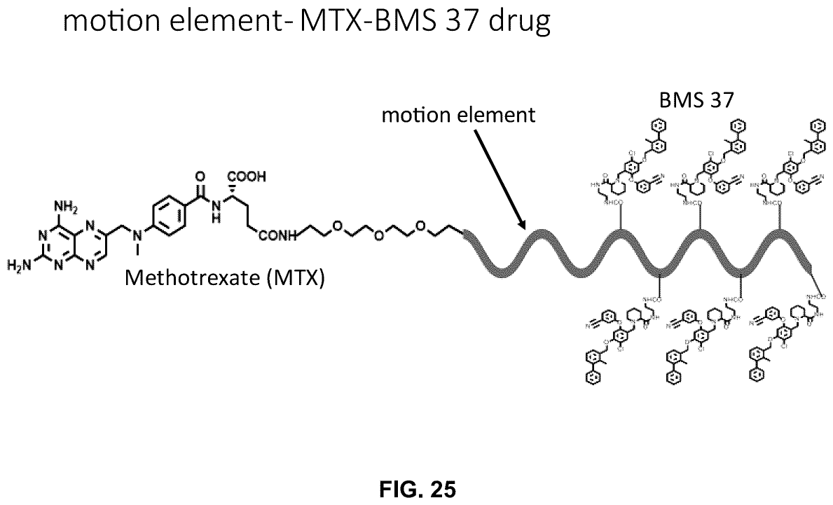

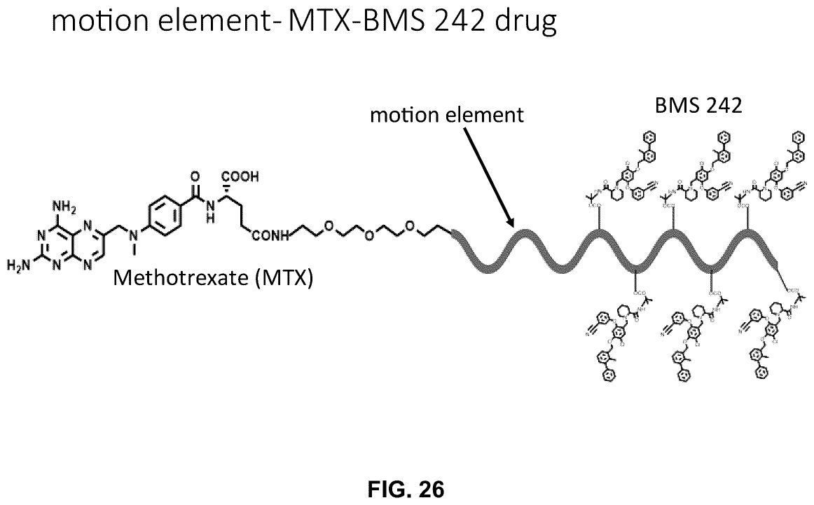

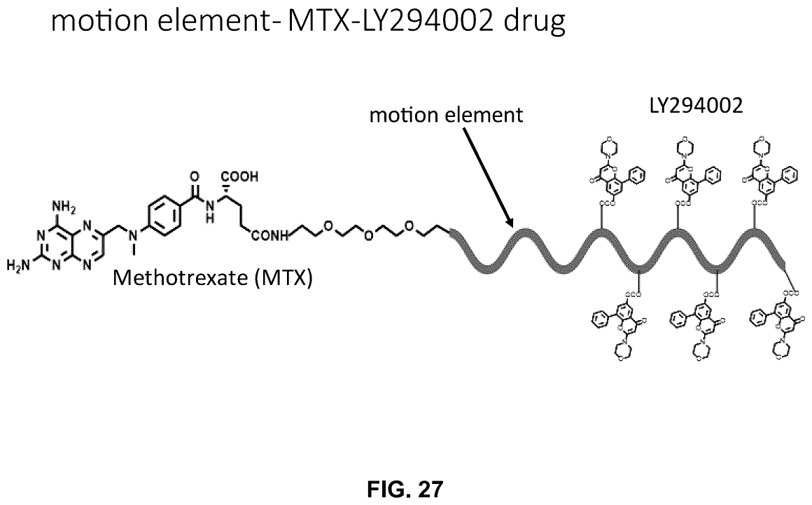

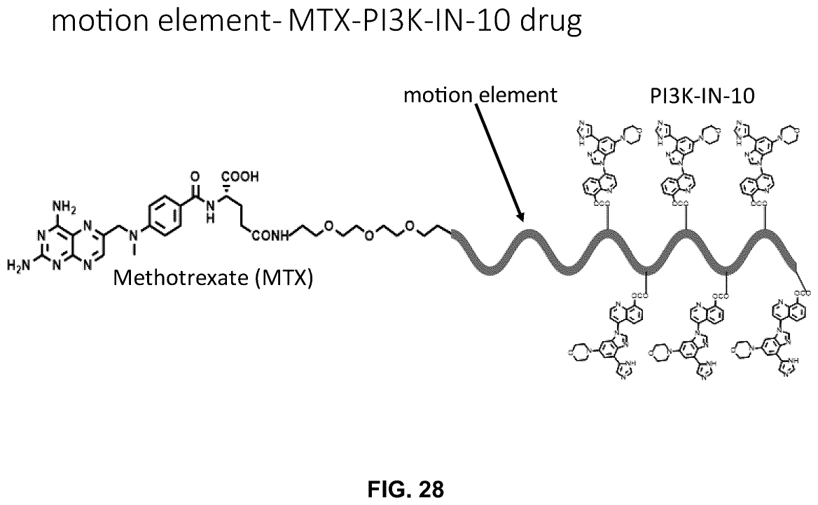

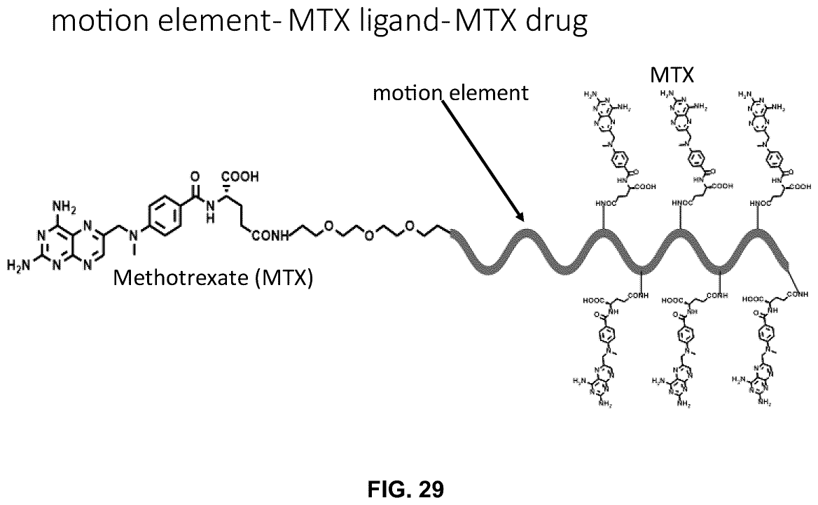

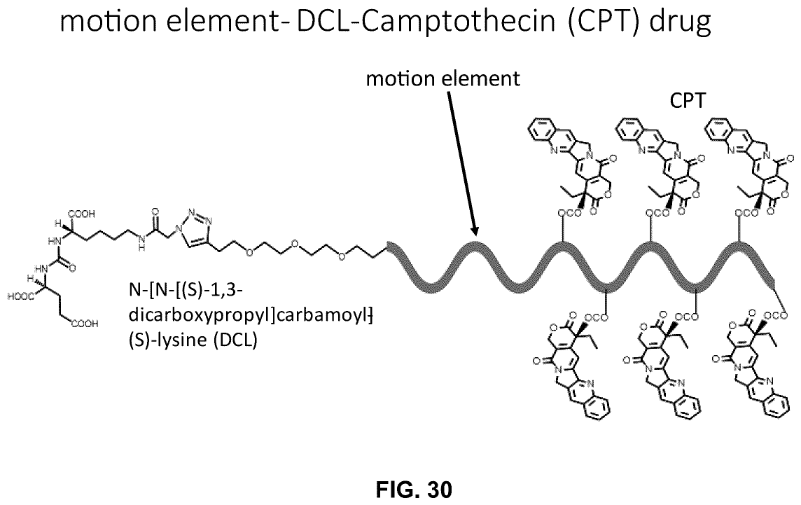

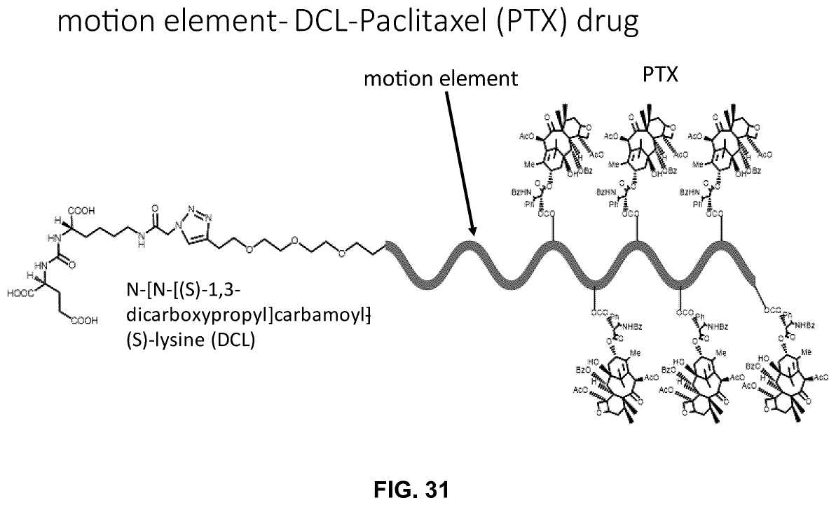

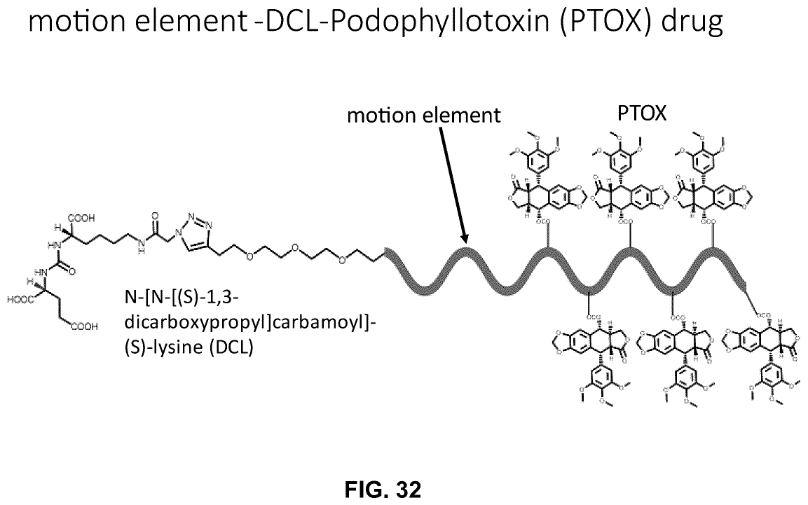

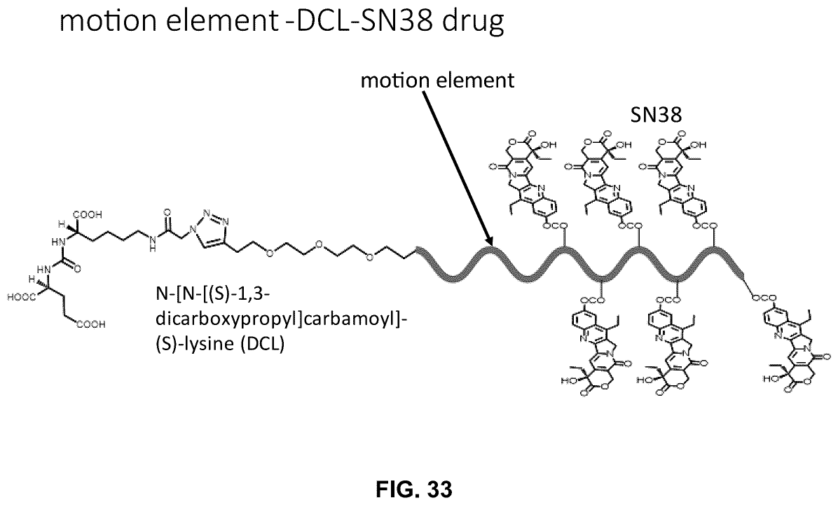

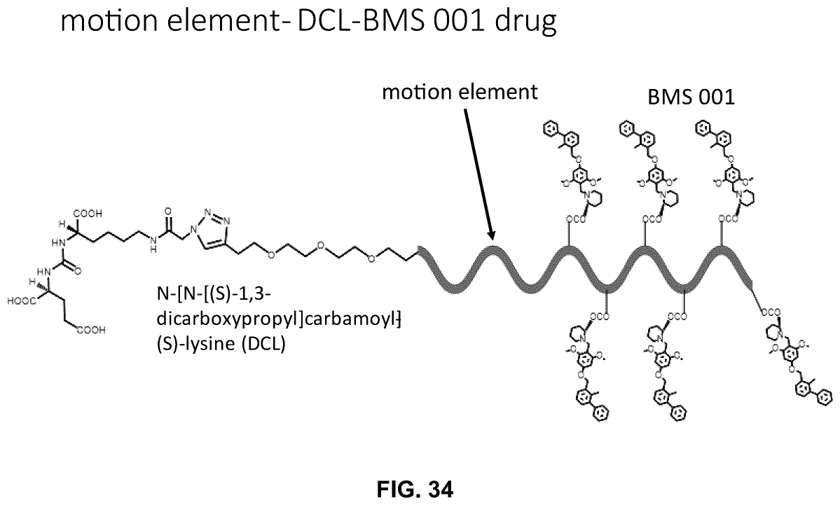

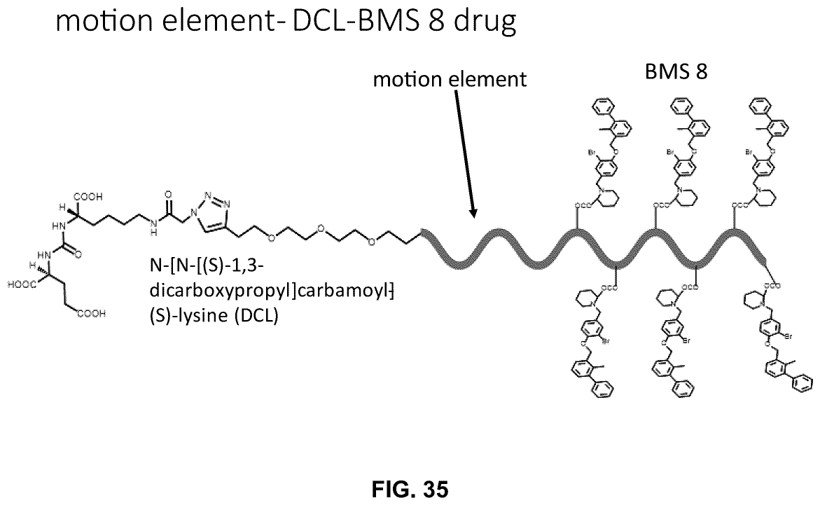

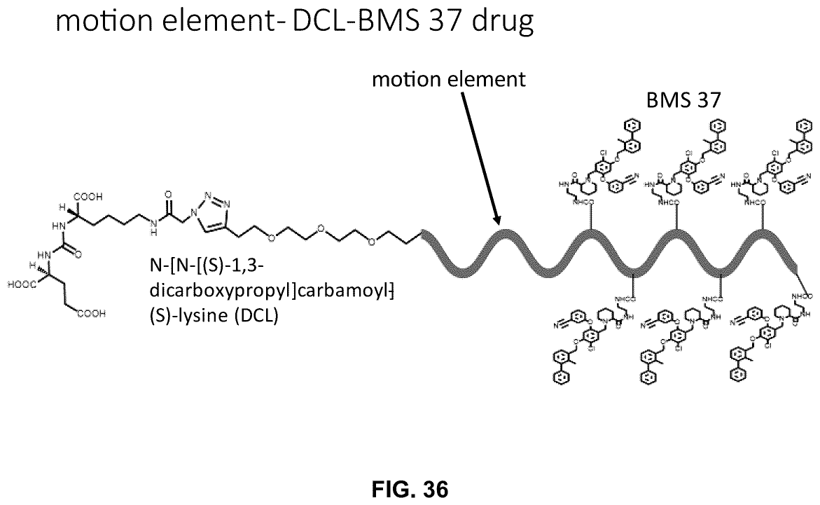

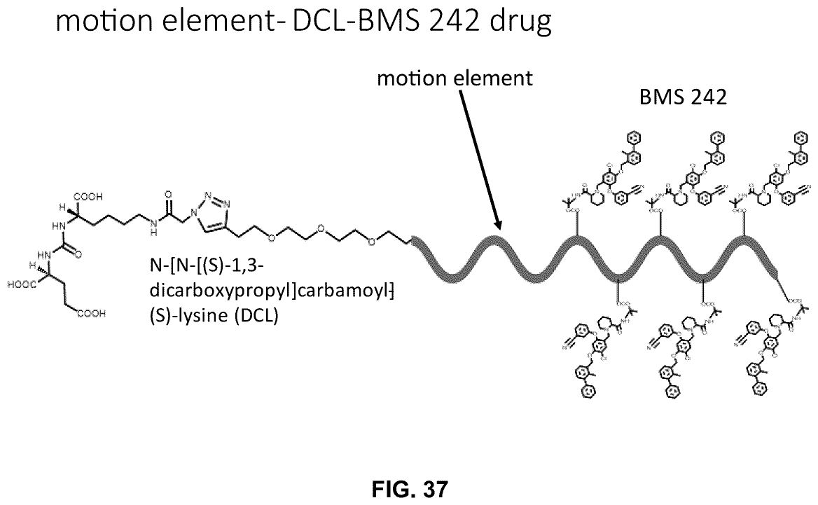

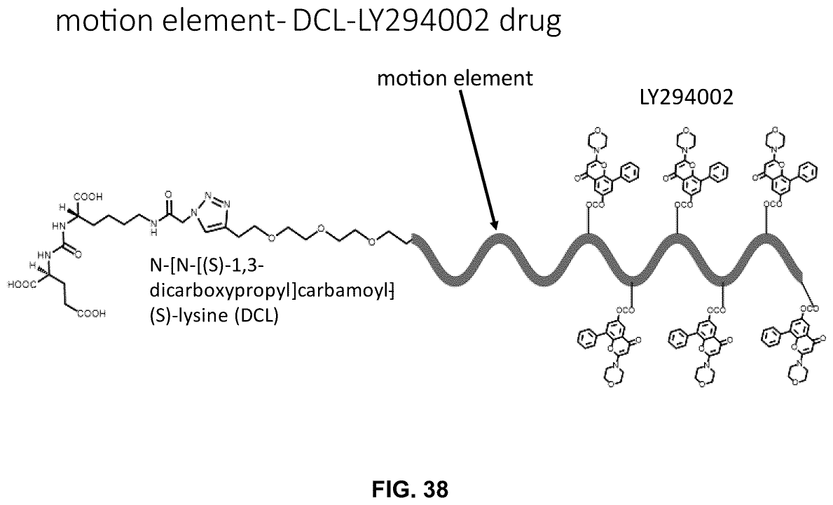

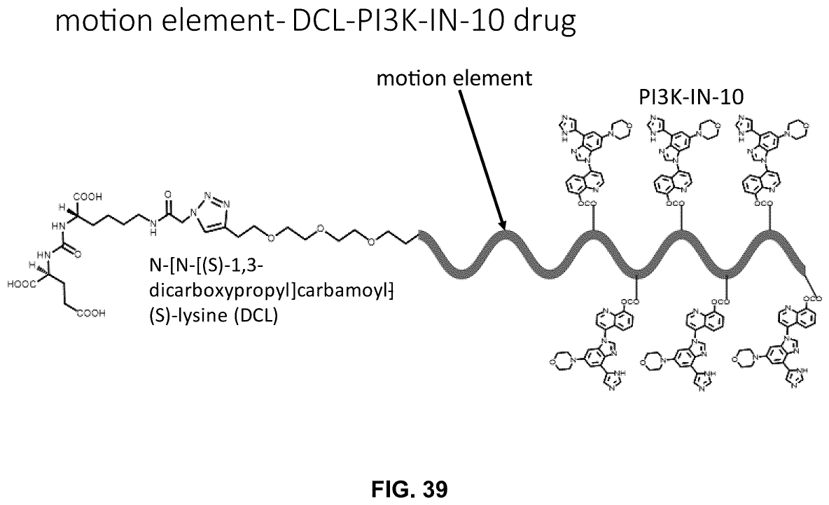

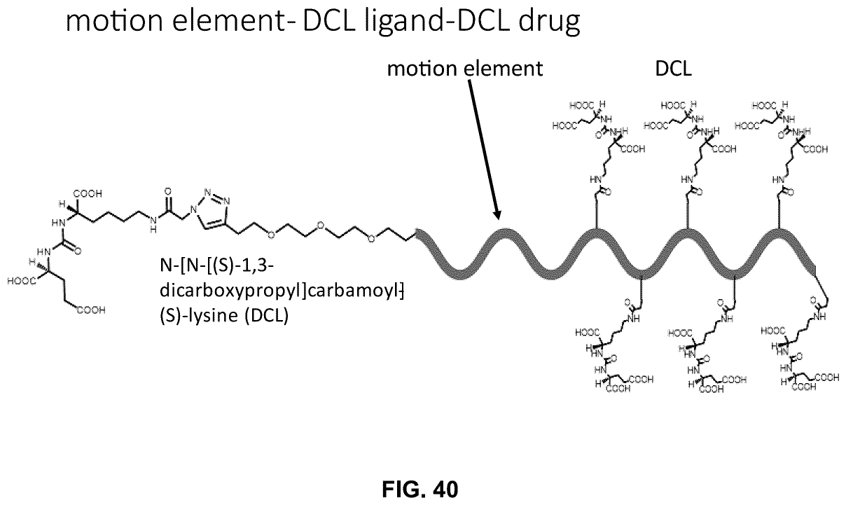

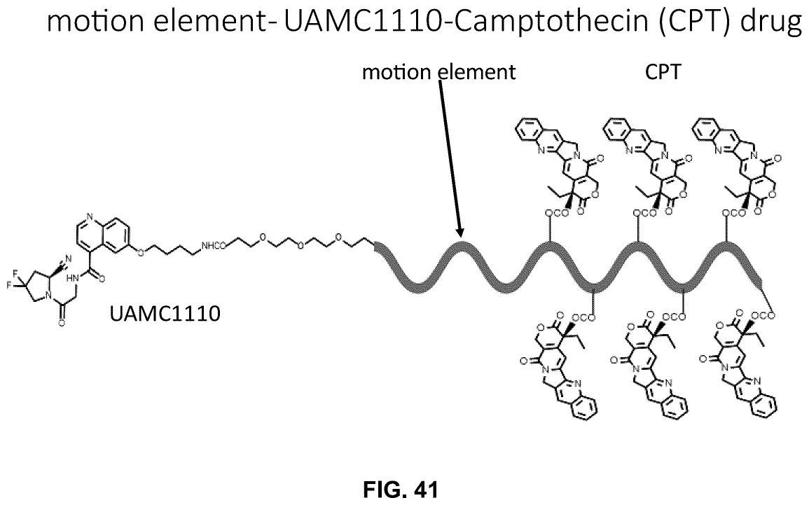

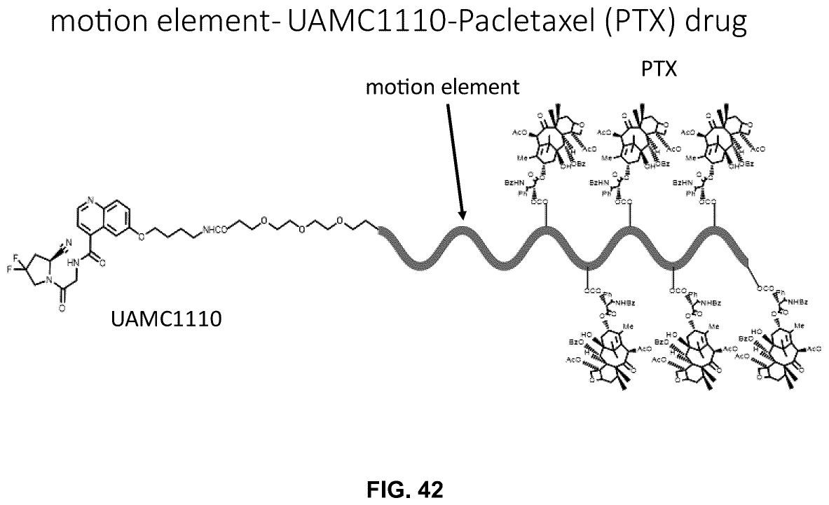

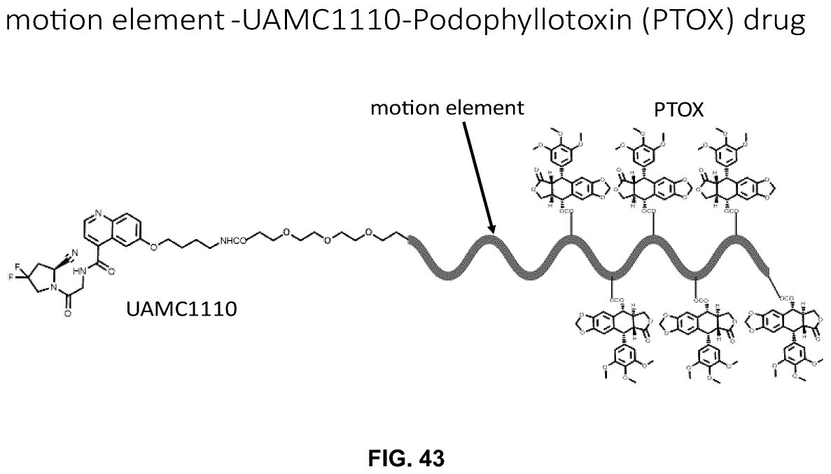

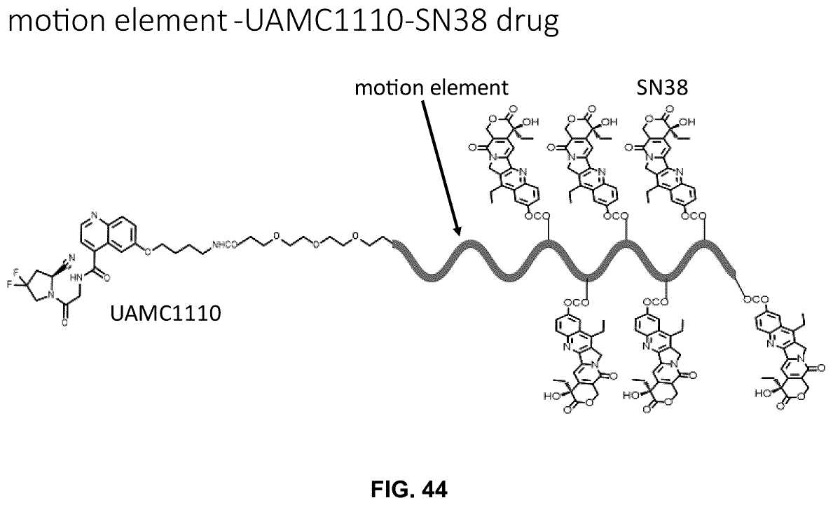

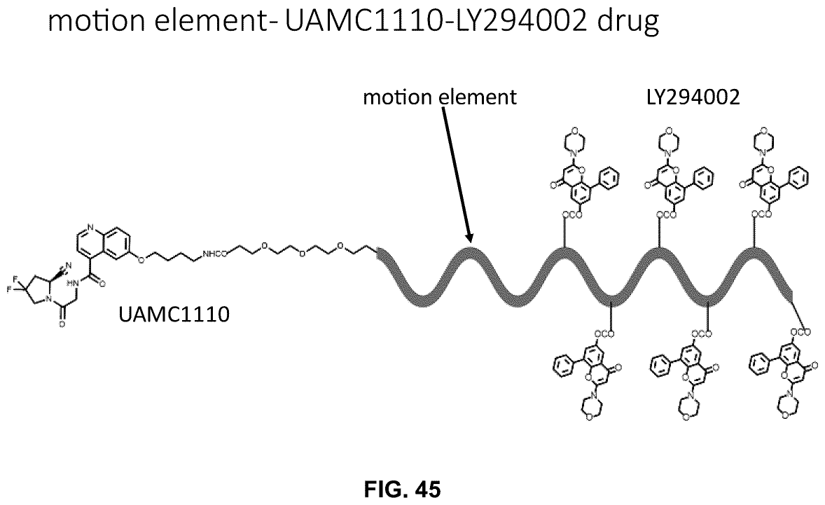

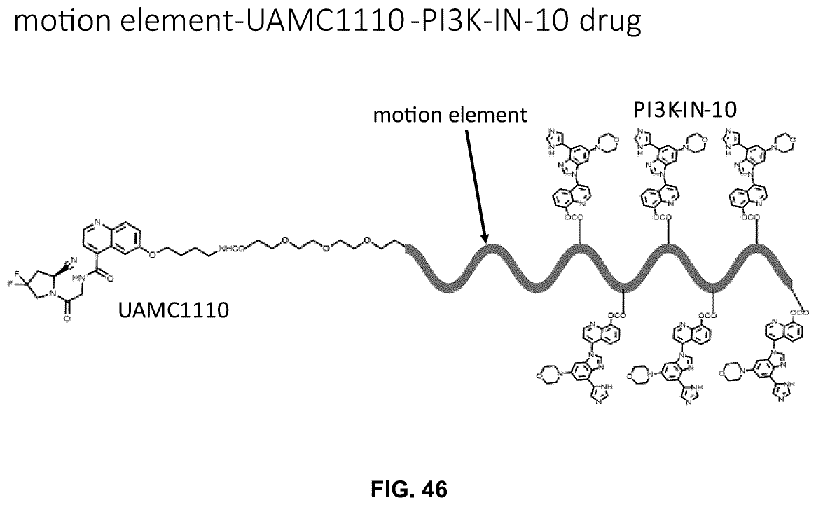

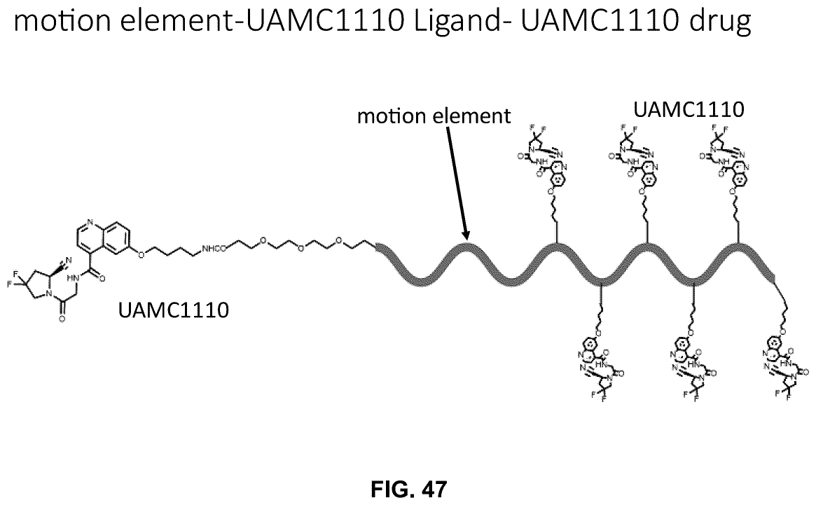

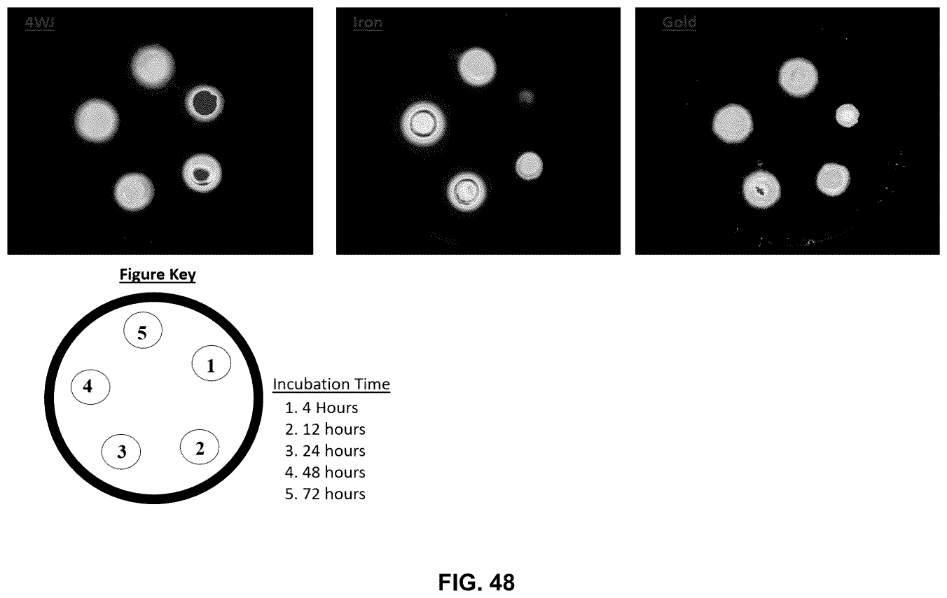

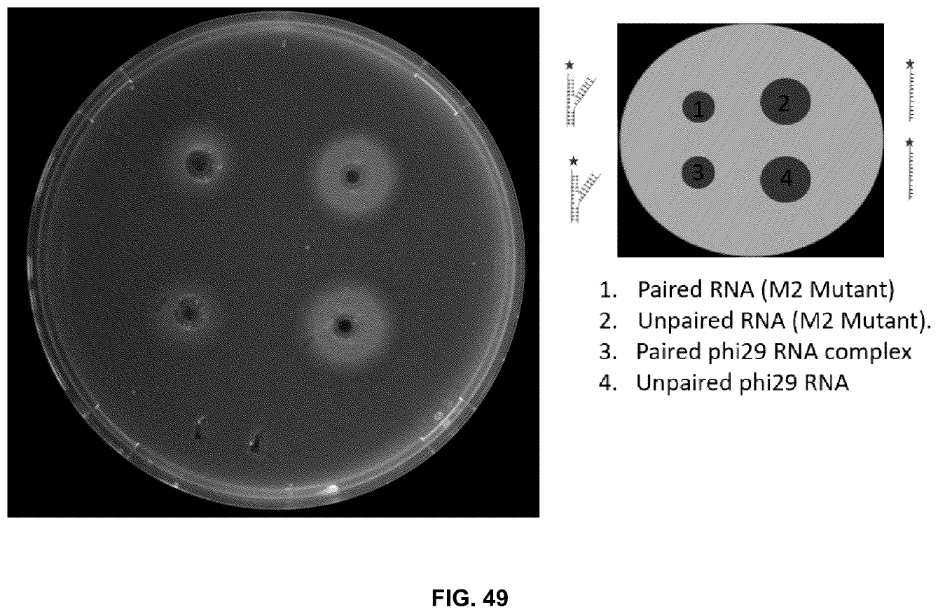

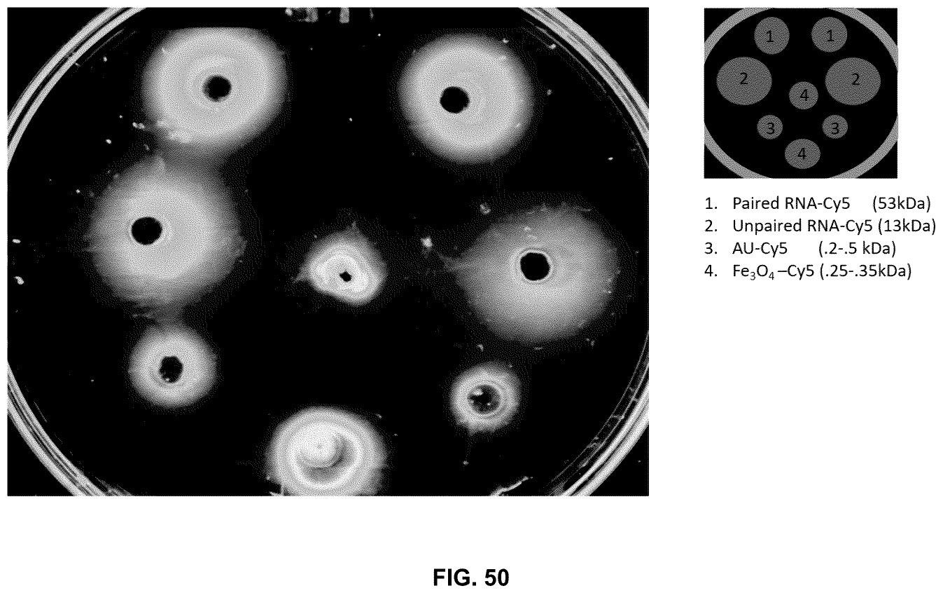

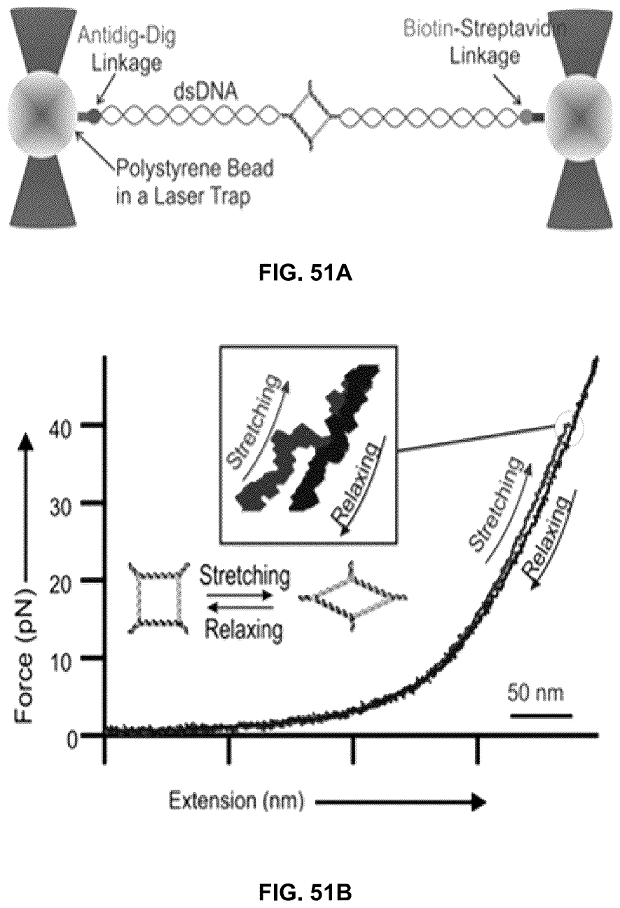

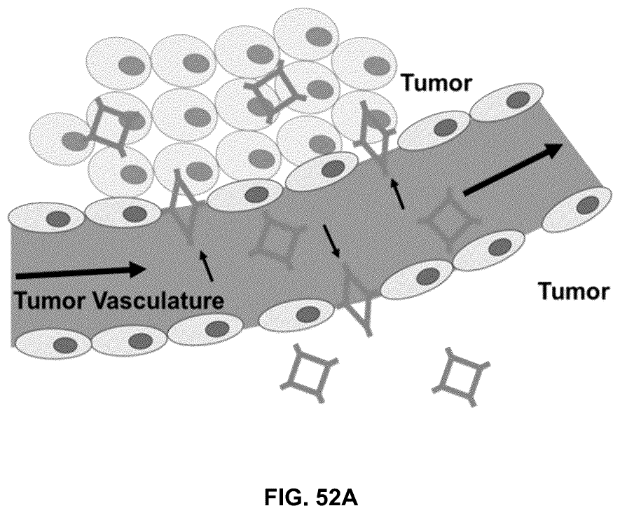

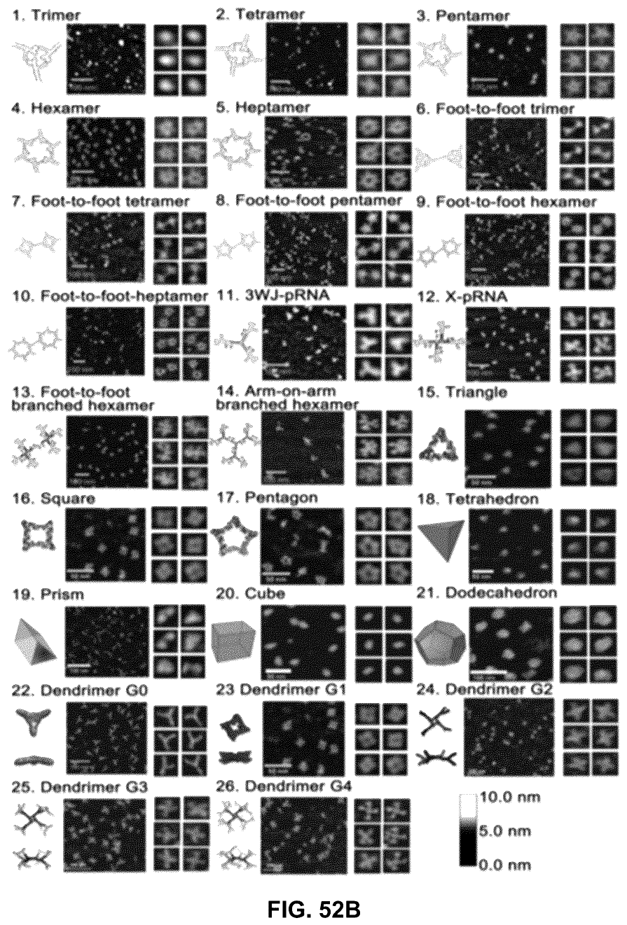

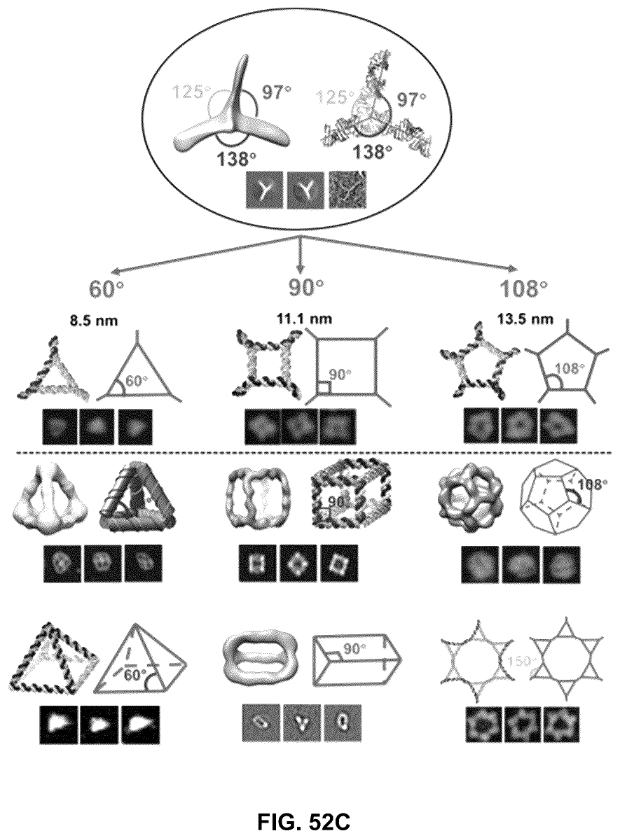

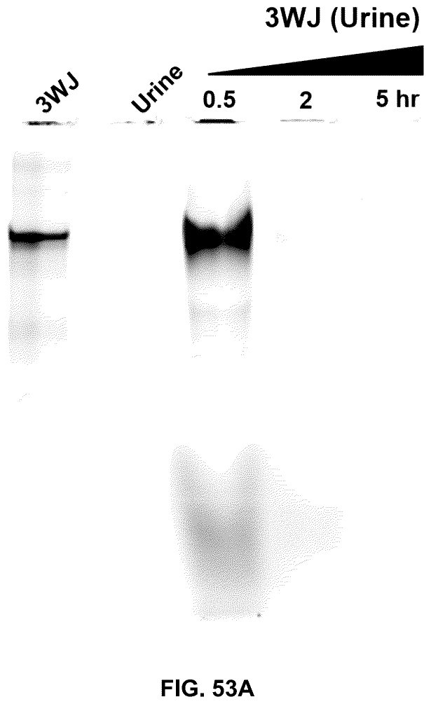

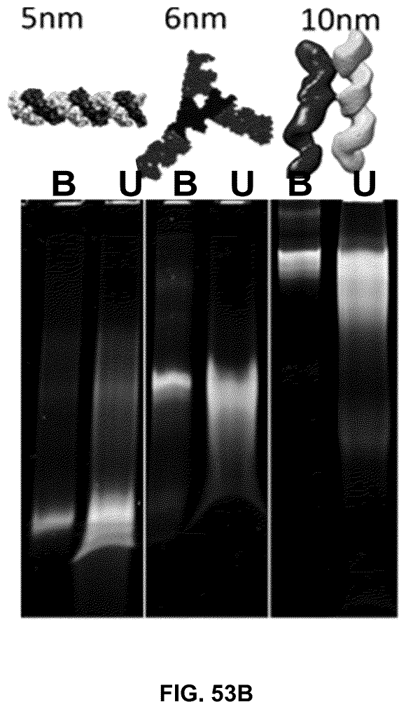

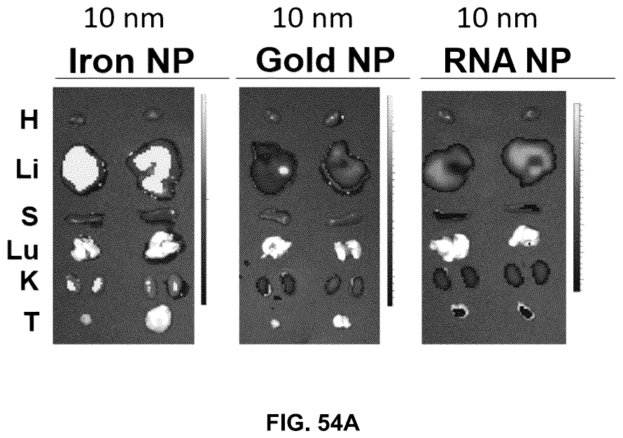

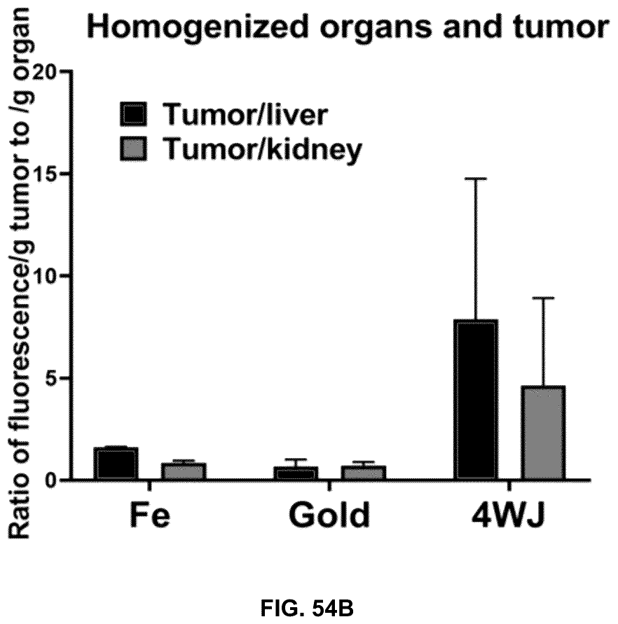

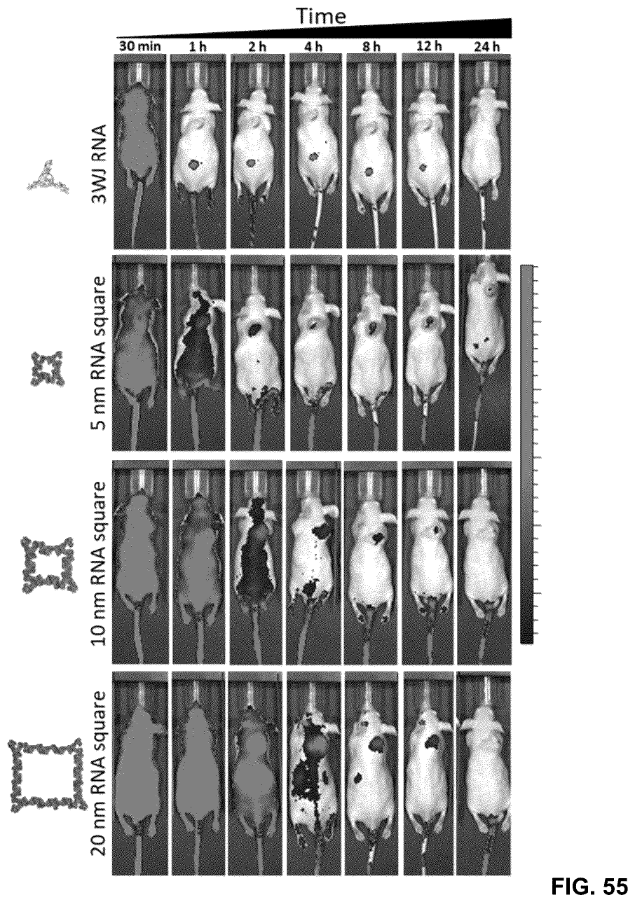

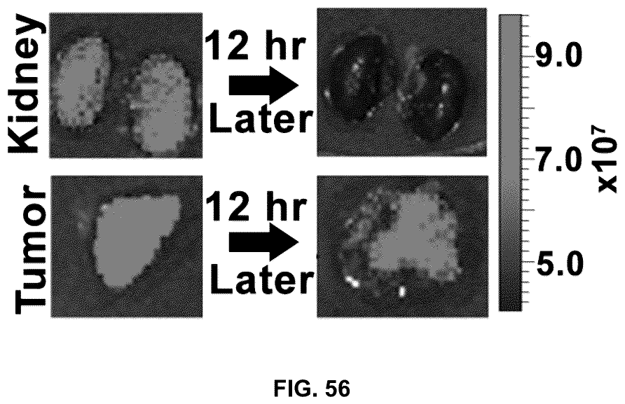

To achieve the PET imaging of prostate cancer using constructed RNA nanoparticles, the radiolabeling of RNA was performed in a “one-pot” self-assembly method. By conjugating 2,2′,2″-(1,4,7-triazacyclononane-1,4,7-triyl)triacetic acid (NOTA) group as radioisotope chelator, the RNA complex was radiolabeled with ⁶⁸Gallium³⁺ (⁶⁸Ga³⁺). To maintain a high chelation efficacy, the reaction temperature and pH was optimized. As shown in FIG. 17A left, radiolabeled RNA complex was analyzed on size-exclusion chromatography. The retention time of [68Ga]-motion element-NOTA was 6.44 minutes, and very low amount of [⁶⁸Ga]Ga³⁺ was detected at 9 min. Similar results were observed for Retention time of [⁶⁸Ga]-motion element-PSMA-NOTA nanoparticles (FIG. 17A, right). The radiochemical purity (RCP) for motion element-NOTA and motion element-PSMA-NOTA was determined as 90% and 91%, respectively. The optimal radiolabeling yield of motion element-NOTA and motion element-PSMA-NOTA was achieved as a function of pH (3.5-4.5) and temperature (10-15 minutes at >95° C.). The results demonstrated the high chelation efficacy of ⁶⁸Ga³⁺ on NOTA conjugated RNA nanoparticles.

In Vivo PET Imaging Using Radiolabeled RNA Nanoparticles

Small PSMA-targeted molecules labeled with 68 Ga and 18F have been developed for primary staging and detection of disease at biochemical recurrence using PET/computed tomography (PET/CT) (Li, R. et al. Prostate Cancer Prostatic Dis. 2018 21:4-21; Maurer, T. et al. Nat Rev Urol. 2016 13:226-235). In this work, we used radiolabeled RNA nanoparticles for in vivo PET/CT imaging of prostate cancer, providing useful information on the prostate cancer staging and therapeutic outcome.

Male BALB/c nude mice bearing PC 3 PIP tumors were injected with [68Ga]-motion element-NOTA (1.8 MBq, 100 μL) and PET imaging was performed at 1 h and 4 h post injection. As shown in FIG. 17B, the radioactivity was highly accumulated in tumor and bladder. At 1 h post-injection, the majority of the radioisotope was observed in bladder and kidney, indicating the rapid renal clearance of the motion element-NOTA. On the images of [⁶⁸Ga]-motion element-NOTA at 4 h post-injection, the PET imaging intensity at tumor was clearly enhanced, suggesting the increased accumulation in PC3 PIP tumor. Moreover, less liver accumulation was observed at 4 h post-injection compared with that at 1 h time point. The PET imaging results demonstrated the specific targeting of RNA nanoparticles to prostate cancer in vivo and showed potentials for guiding the therapy of prostate cancer.

Discussions

PET image-guided therapy provided a promising paradigm for prostate cancer treatment in clinic. Traditional radioligand targeting PSMA offered sensitive prostate cancer imaging and effective radiotherapy (Lenzo, N. P. et al. Diagnostics (Basel). 2018 8; Corfield, J. et al. World J Urol. 2018 36:519-527). However, as the recent cancer treatment was impeded by resistance and low drug delivery efficacy, development of a multi-functional nanoscale delivery system is imperative. Here we reported the construction of motion element-based RNA nanoparticles with controllable valency of targeting ligands. The DCL ligand has been conjugated on RNA nanoparticles with mono-, bi- or trivalent presentation, making it possible to control the interaction with PSMA overexpressed prostate cancer cells. In vitro flow cytometry and confocal microscopy imaging results demonstrated the exceptional targeting capability of bivalent and trivalent DCL conjugated RNA nanoparticles. Further in vivo fluorescent imaging in PC3 PIP xenograft bearing mice proved the specific targeting to tumor with a higher accumulation in tumor compared to motion element-PSMA nanoparticle. By radiolabeling of RNA nanoparticles, we were able to perform in vivo PET imaging of prostate cancer with high sensitivity. Results showed the enhancement of radiosignal at the tumor over time within the first 4 hours post-injection, indicating the accumulation of RNA nanoparticles in tumor.

Methods

Conjugation of Multivalent DCL on RNA

DCL-azide was conjugated to alkyne-modified RNA oligos via Copper-catalyzed azide-alkyne cycloaddition (CuAAC). 100 mM CuBr solution (tBuOH:DMSO=1:3) was mixed with 100 mM TBTA (tBuOH:DMSO=1:3) in a ratio of 1:2 to yield Cu-TBTA complex. 10 μL of such freshly prepared Cu-TBTA solution was added to 60 μL DCL-azide solution (DMSO, 20 mM) followed by adding 50 μL alkyne-modified RNA oligos solution (water, 1-2.5 mM measured by absorbance at 260 nm). The reaction was completed overnight at room temperature. Water was added to reach 400 μL (total volume) followed by adding 40 μL 3 M sodium acetate (pH 5.2) and 1100 μL ethanol. The tube was stored at −20° C. for at least two hours to precipitate RNA-DCL conjugate. After centrifugation at maximal speed (13100 RPM) at 4° C. for 30 minutes and washing the pellet with 70% cold ethanol, the pellet was dried by speed vacuum. 100 μL water was used to resuspend RNA-DCL conjugate. RNA-DCL conjugate was further purified by reverse phase HPLC (Solvent A: 0.1 M triethylamine acetate (TEAA) in water and solvent B: 75% acetonitrile and 25% water with 0.1 M TEAA).

NOTA and Fluorophore Conjugation on RNA

NOTA NHS ester was purchased from CheMatech and AFdye 647 NHS ester was purchased from Click Chemistry Tools. Conjugation reactions were carried out by mixing a 1:10 molar ratio of primary amine labeled motion element:NHS ester in 0.1M sodium bicarbonate buffer, pH=8.5. The conjugation reactions were incubated at room temperature for 16 hours while protected from light. Following incubation, the reactions were ethanol precipitated and washed twice with cold 75% ethanol to remove the unreacted NOTA or fluorophore, facilitating purification by HPLC.

Reverse-Phase HPLC Purification of RNA-AFdye 647 Strand

Fluorophore conjugated RNA was purified using ion-pairing reverse phase (IPRP) HPLC. Gradient mobile phase was used to separate dye-modified RNA from unmodified strands. Buffer A was 0.1 M TEAA (Glen Research) in water and buffer B was 0.1 M TEAA in 75% acetonitrile and 25% water. All purifications were performed on Agilent PLRP-S column (Agilent Cat. #: PL1512-5500) in Agilent 1260 Infinity HPLC system. A flow rate of 1 mL/min was used throughout all HPLC methods and absorbance was monitored at 260 nm (RNA) and 650 nm (Cy5). Fractions were collected and dried under vacuum and then resuspended in DEPC water. 8M urea 16% denaturing polyacrylamide gel electrophoresis (PAGE) was used to characterize the purified strands.

Size-Exclusion Chromatography HPLC Purification of NOTA-Conjugated RNA Strand

NOTA conjugated RNA was purified from unreacted NOTA using size-exclusion HPLC. HPLC grade water was used as the mobile phase. All purifications were performed on Agilent Bio-SEC column in Agilent 1260 Infinity HPLC system. A flow rate of 0.3 mL/min was used and absorbance was monitored at 260 nm and 305 nm for RNA and NOTA, respectively. Fractions were collected and dried under vacuum and resuspended in diethyl pyrocarbonate (DEPC) treated water.

RNA Nanoparticle Assembly and Purification

Equimolar ratio of composite strands was mixed in Tris Acetate buffer. By denaturing the strands at 95° C., the mixture solution was slowly cooled down to 4° C. at the rate of −2° C./min. The assembled nanoparticles were desalted by Sephadex G50 column to remove salts and small molecule contaminants.

Radiolabeling of RNA Nanoparticles and Chelation Efficacy Determination

1.0 ml of 1.4 mCi ⁶⁸GaCl₃ (in 0.1 M HCl) was taken in a glass reaction vial; and pH was adjusted to ˜3.6 using 1 M NaOAc buffer. 200 μg of the RNA nanoparticles was added and the reaction mixture was concentrated down to 0.5 ml using a combination of heat (90-95° C.) and nitrogen flow, raising the pH slowly to ˜5-5.5 during this time using NaOAc buffers. Aliquoted radiolabeled sample was then injected into size-exclusion chromatography (SEC) HPLC to determine the radiolabeling efficacy. Agilent Bio SEC-3 100 Å 4.6×300 mm was used with 0.15M NaCl as mobile phase. UV absorbance at 260 nm and a radioisotope detector were used to monitor the RNA and 68Ga3+. Free 68Ga3+ came off the column at around 9 min while RNA complex was eluted at around 6 min.

Temperature Gradient Gel Electrophoresis (TGGE) Characterization

Preassembled RNA nanoparticles were run in a 10% (w/v) native PAGE in Tris-borate-EDTA buffer at 100 V for 10 min at room temperature. Temperature gradient gel electrophoresis (TGGE) analysis was then performed in a buffer containing 50 mM TRIS at pH=8.0, 100 mM NaCl, and 0.2 mM MgCl₂ as previously reported. A gradient temperature (40-80° C.) was applied perpendicular to the electrical current, and the experiment was run for 1 h at 20 W. The gel was stained and visualized as described above. Quantified values of bands for each nanoparticle were divided by the sum of the total values in corresponding lanes, calculated by ImageJ. Melting curves were plotted with quantified data points using GraphPad Prism 8. Tm values were defined as the temperature at which 50% of the loaded nanoparticles dissociated.

DLS Characterization

The apparent hydrodynamic diameter and zeta potential of the RNA nanoparticles in 1 μM in TMS buffer were determined using a Zetasizer Nano-ZS (Malvern Instrument) at 25° C. The laser wavelength was 633 nm. Results were plotted with data points using GraphPad Prism 8 software. Three independent runs with 8 measurements each run were recorded.

Serum Stability Characterization of RNA Motion Element-TriDCL and Motion Element-PSMA Samples

Assembled RNA nanoparticles were incubated in cell culture medium containing 10% (v/v) FBS at a final concentration of 1 μM at 37° C. After removal from 37° C. incubation at their respective time points, the samples were frozen at −80° C. to prevent any further degradation. The resulting samples were examined on 6% native PAGE followed by fluorescent imaging by Typhoon imaging system under ethidium bromide and Cy5 channel. Quantification analysis was performed using ImageJ software and plotted by GraphPad Prism 8 software.

Cell Culture

PC3 and PC3 PIP cells were obtained from American Type Culture Collection (ATCC). KB cells were grown and cultured in 1640 RPMI medium (Thermo Fisher Scientific) containing 10% (v/v) FBS at 37° C. in humidified air environment and 5% CO2.

Flow Cytometry Analysis

100 nM RNA nanoparticles with varying number of DCL harboring AFDye 647 were each incubated with 2×10⁵ PC3 and PC3 PIP cells at 37° C. for 1 h. After washing with phosphate-buffered saline (PBS) twice, the cells were resuspended in PBS. Flow cytometry analysis was performed by The Ohio State University Analytical Cytometry Shared Resource (ACSR) core facility to compare the binding efficacy of the fluorophore labeled RNA nanoparticles with different cells. The data was analyzed by FlowJo 7.6.1 software.

Confocal Microscopy Imaging

PC3 and PC3 PIP cells were seeded on glass cover slips and cultured at 37° C. overnight. AFDye 647 labeled RNA nanoparticles were incubated with cells at a final concentration of 100 nM for 1 h at 37° C. The slips were washed twice with PBS buffer followed by fixation using 4% formaldehyde. 0.1% Triton X-100 (Sigma-Aldrich) in PBS buffer was used to treat the slips for 5 min, followed by subsequent cytoskeleton staining using Alexa Fluor 488 phalloidin (Thermo Fisher Scientific) for 30 min at room temperature. After rinsing with PBS buffer, the cells were mounted with ProLong@ Gold Antifade Reagent (Life Technologies Corp.) containing 4′,6-diamidino-2-phenylindole (DAPI) for cell nucleus staining and imaged on Olympus FV3000 confocal microscope (Olympus Corp.). Data were acquired using Fluoview FV31S-SW.

In Vivo Biodistribution Study by Fluorescent Imaging

80 μL of 15 μM RNA nanoparticles fluorescently labeled with AFDye 647 were injected into 5-6 weeks old PC3 PIP tumor bearing male BALB/c mice (Taconic) intravenously through the tail vein. Mice administered with PBS served as blank controls. At 8 h post-injection, mice were sacrificed, and their hearts, kidneys, livers, spleen, and lungs were collected and imaged for Cy5 fluorescent signal using an In Vivo Imaging System (IVIS) imager (Caliper Life Sciences). All animal experiments were housed and performed in accordance with the Subcommittee on Research Animal Care of the Ohio State University guidelines approved by the Institutional Review Board.

In Vivo PET/CT Imaging

To visualize the in vivo performance of Motion element-NOTA/68Ga, small animal PET/CT imaging was applied using PC3 PIP xenograft-bearing male nude mice model. Mice were immobilized on the scanner bed (orientation: prone, nose first) using a 1.5% to 2.5% isoflurane/anaesthetic air mixture for the duration of the scans and were monitored for body temperature and breathing rate. CT X-ray images were acquired for anatomical reference and to allow for scatter and attenuation correction during PET data reconstruction, using the following parameters: 50 KeV, 200 ms, 1:5 binning, and a matrix size of 125×125×125 μm. The ex vivo biodistribution study employed administration of Motion element-NOTA/⁶⁸Ga in mice with the lowest injected dose of 50 μCi per mice by tail vein injection. Micro PET image data were acquired at 1 h, 2 h, 3 h and 4 h post-injection.

Synthesis of DCL-azido

To a solution of DCL (8 mg) in DMSO (250 μL) was added N,N-diisopropylethylamine (0.08 mL, 0.47 mmol), followed by NHS-azido (7 mg). After incubation at room temperature for 4 h, the reaction mixture was directly used for conjugation with alkyne-modified RNA.

Synthesis of RNA Oligos

Solid-phase RNA oligomer synthesis was performed on a 1 μmole scale. An automated oligo synthesizer ASM-800E from Biosset was used to start the synthesis from the universal 1,000 Å long chain amino alkyl-controlled pore glass (LCAA-CPG) solid support. Coupling efficiency was monitored after removal of the dimethoxytrityl (DMT) protecting groups. Sequences of the RNA oligomers used were listed in 5′ to 3′ orientation (“r” denotes 2′-OH base, “f” denotes 2′-F modified base). Each strand was synthesized using 2′-fluorinated pyrimidine. Following synthesis, oligomers were cleaved from beads and deprotected in a 1:1 mixture (v/v) of ammonium hydroxide and methylamine (AMA) solution at RT for 2 h. The 2′-TBDMS protecting groups were removed by triethylamine trihydrofluoride (TEA·3HF) followed by desalting using Glen gel-pak desalting column. The collected fraction was dried under speed vacuum. Then the synthesized strands were analyzed using 8 M urea 16% denaturing polyacrylamide gel electrophoresis (PAGE).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A therapeutic structure containing
a RNA nanostructure motion element,
a methotrexate (MTX), N—[N—[(S)-1,3-dicarboxypropyl]carbamoyl]-(S)-lysine (DCL), or UAMC-1110 ligand, and
a plurality of drug molecules selected from the group consisting of camptothecin (CPT), paclitaxel (PTX), podophyllotoxin (PTOX), 7-Ethyl-10-hydroxycamptothecin (SN38), BMS1, BMS8, BMS27, BMS242, LY294002, PI3K-IN-20, and methotrexate (MTX).

2. The therapeutic structure of claim 1, wherein the RNA nanostructure motion element comprises at least three synthetic RNA oligonucleotides,
wherein the at least three synthetic RNA oligonucleotides are coupled to each other to form a central core domain and at least three double-stranded arms arranged around the core domain and extending away from the central core domain,
wherein the MTX, DCL, or UAMC-1110 ligand is conjugated to one of the at least three synthetic RNA oligonucleotides,
wherein the plurality of drug molecules are conjugated to the at least three synthetic RNA oligonucleotides, and
wherein the at least three synthetic RNA oligonucleotides are configured to self-assemble to form the RNA nanostructure.

3. The therapeutic structure of claim 2, comprising at least 2, 3, 4, 5, 6, 7, 8, 9, 10, 11, 12, 13, 14, 15, 16, 17, 18, 19, 20, 25, 30, 35, 40, 45, 50 drug molecules conjugated to the at least three synthetic RNA oligonucleotides.

4. The therapeutic structure of claim 2, wherein the MTX, DCL, or UAMC-1110 ligand is conjugated to a terminal nucleotide.

5. The therapeutic structure of claim 2, wherein the plurality of drug molecules are conjugated to a non-terminal nucleotide.

6. The therapeutic structure of claim 2, wherein the Tm of the therapeutic structure is at least 60 degrees Celsius.


SEQUENCE LISTING (5 sequences)

SEQ ID NO: 1 - RNA, 39 nt, synthetic construct
  source 1..39: mol_type = other RNA
  modified_base 1..3: mod_base = OTHER; note = 2-F nucleotide
  modified_base 5..7: mod_base = OTHER; note = 2-F nucleotide
  modified_base 11: mod_base = OTHER; note = 2-F nucleotide
  modified_base 13: mod_base = OTHER; note = 2-F nucleotide
  modified_base 15: mod_base = OTHER; note = 2-F nucleotide
  modified_base 17..18: mod_base = OTHER; note = 2-F nucleotide
  modified_base 21: mod_base = OTHER; note = 2-F nucleotide
  modified_base 23..24: mod_base = OTHER; note = 2-F nucleotide
  modified_base 26..27: mod_base = OTHER; note = 2-F nucleotide
  modified_base 30: mod_base = OTHER; note = 2-F nucleotide
  modified_base 32: mod_base = OTHER; note = 2-F nucleotide
cctattcaggtgcgtgctggtgctaccgatgtaattcaa

SEQ ID NO: 2 - RNA, 44 nt, synthetic construct
  source 1..44: mol_type = other RNA
  modified_base 1..4: mod_base = OTHER; note = 2-F nucleotide
  modified_base 6..8: mod_base = OTHER; note = 2-F nucleotide
  modified_base 10..12: mod_base = OTHER; note = 2-F nucleotide
  modified_base 16: mod_base = OTHER; note = 2-F nucleotide
  modified_base 18: mod_base = OTHER; note = 2-F nucleotide
  modified_base 20: mod_base = OTHER; note = 2-F nucleotide
  modified_base 22..23: mod_base = OTHER; note = 2-F nucleotide
  modified_base 26: mod_base = OTHER; note = 2-F nucleotide
  modified_base 28..29: mod_base = OTHER; note = 2-F nucleotide
  modified_base 31..32: mod_base = OTHER; note = 2-F nucleotide
  modified_base 35: mod_base = OTHER; note = 2-F nucleotide
  modified_base 37: mod_base = OTHER; note = 2-F nucleotide
  modified_base 40..42: mod_base = OTHER; note = 2-F nucleotide
tttttcctattcaggtgcgtgctggtgctaccgatgtaattcaa

SEQ ID NO: 3 - RNA, 44 nt, synthetic construct; identical to SEQ ID NO: 2
  source 1..44: mol_type = other RNA
  modified_base 1..4: mod_base = OTHER; note = 2-F nucleotides
  modified_base 6..7: mod_base = OTHER; note = 2-F nucleotides
  modified_base 11: mod_base = OTHER; note = 2-F nucleotides
  modified_base 13: mod_base = OTHER; note = 2-F nucleotides
  modified_base 15: mod_base = OTHER; note = 2-F nucleotides
  modified_base 17..18: mod_base = OTHER; note = 2-F nucleotides
  modified_base 21: mod_base = OTHER; note = 2-F nucleotides
  modified_base 23..24: mod_base = OTHER; note = 2-F nucleotides
  modified_base 26: mod_base = OTHER; note = 2-F nucleotides
  modified_base 32: mod_base = OTHER; note = 2-F nucleotides
  modified_base 36: mod_base = OTHER; note = 2-F nucleotides
  modified_base 8: mod_base = OTHER; note = 2-propargyl nucleotide
  modified_base 10: mod_base = OTHER; note = 2-propargyl nucleotide
  modified_base 12: mod_base = OTHER; note = 2-propargyl nucleotide
  modified_base 32: mod_base = OTHER; note = 2-propargyl nucleotide
  modified_base 35: mod_base = OTHER; note = 2-propargyl nucleotide
  modified_base 40: mod_base = OTHER; note = 2-propargyl nucleotide
  modified_base 42: mod_base = OTHER; note = 2-propargyl nucleotide

SEQ ID NO: 4 - RNA, 41 nt, synthetic construct
  source 1..41: mol_type = other RNA
  modified_base 1: mod_base = OTHER; note = 2-F nucleotide
  modified_base 7: mod_base = OTHER; note = 2-F nucleotide
  modified_base 11..12: mod_base = OTHER; note = 2-F nucleotide
  modified_base 15: mod_base = OTHER; note = 2-F nucleotide
  modified_base 18: mod_base = OTHER; note = 2-F nucleotide
  modified_base 20: mod_base = OTHER; note = 2-F nucleotide
  modified_base 24..25: mod_base = OTHER; note = 2-F nucleotide
  modified_base 34: mod_base = OTHER; note = 2-F nucleotide
  modified_base 2: mod_base = OTHER; note = 2-propargyl nucleotide
  modified_base 6: mod_base = OTHER; note = 2-propargyl nucleotide
  modified_base 9: mod_base = OTHER; note = 2-propargyl nucleotide
  modified_base 27: mod_base = OTHER; note = 2-propargyl nucleotide
  modified_base 29: mod_base = OTHER; note = 2-propargyl nucleotide
  modified_base 35: mod_base = OTHER; note = 2-propargyl nucleotide
  modified_base 39: mod_base = OTHER; note = 2-propargyl nucleotide
ttgaattacatcggtagcacgggctgtgcgaggctgaacag

SEQ ID NO: 5 - RNA, 43 nt, synthetic construct
  source 1..43: mol_type = other RNA
  modified_base 1..4: mod_base = OTHER; note = 2-F nucleotides
  modified_base 6: mod_base = OTHER; note = 2-F nucleotides
  modified_base 10: mod_base = OTHER; note = 2-F nucleotides
  modified_base 14..17: mod_base = OTHER; note = 2-F nucleotides
  modified_base 19: mod_base = OTHER; note = 2-F nucleotides
  modified_base 21: mod_base = OTHER; note = 2-F nucleotides
  modified_base 24..25: mod_base = OTHER; note = 2-F nucleotides
  modified_base 28: mod_base = OTHER; note = 2-F nucleotides
  modified_base 7: mod_base = OTHER; note = 2-propargyl nucleotide
  modified_base 9: mod_base = OTHER; note = 2-propargyl nucleotide
  modified_base 11: mod_base = OTHER; note = 2-propargyl nucleotide
  modified_base 30: mod_base = OTHER; note = 2-propargyl nucleotide
  modified_base 32: mod_base = OTHER; note = 2-propargyl nucleotide
  modified_base 34: mod_base = OTHER; note = 2-propargyl nucleotide
  modified_base 36: mod_base = OTHER; note = 2-propargyl nucleotide
  modified_base 40: mod_base = OTHER; note = 2-propargyl nucleotide
tttttctgttcagcctcgcacagccagcacgcacctgaatagg